(19) United States
(12) Patent Application Publication
(10) Pub. No.: US 2026/0284365 A1
(43) Pub. Date: Sep. 24, 2026

(54) EXPANDABLE ACCESS SHEATH

(71) Applicant: BOSTON SCIENTIFIC SCIMED, INC., Maple Grove, MN (US)

(72) Inventors: Victor Kotov, St. Louis Park, MN (US); Nicholas Lee Tassoni, Andover, MN (US)

(73) Assignee: BOSTON SCIENTIFIC SCIMED, INC., MAPLE GROVE, MN (US)

(21) Appl. No.: 19/566,309

(22) Filed: Mar. 13, 2026

Related U.S. Application Data
(60) Provisional application No. 63/867,193, filed on Aug. 20, 2025.
(60) Provisional application No. 63/775,508, filed on Mar. 21, 2025.

(51) Int. Cl. A61M 25/06; A61M 25/00; A61M 60/865

(52) U.S. Cl. CPC A61M 25/0668; A61M 25/0023; A61M 25/0097; A61M 60/865; A61M 2025/0024

(57) ABSTRACT

Example access sheaths are disclosed. An example access sheath for a medical device includes a hub attached to an elongate shaft, wherein the elongate shaft includes a proximal portion and an expandable portion. Further, the expandable portion is configured to shift between an unexpanded configuration and an expanded configuration. Further, the proximal portion includes a first diameter. Further, the expandable portion includes a second diameter in the unexpanded configuration, and wherein the second diameter is less than the first diameter.

### CROSS-REFERENCE TO RELATED APPLICATIONS

This application claims the benefit of U.S. Patent Application Serial No. 63/775,508, filed Mar. 21, 2025, entitled "REMOVABLE ACCESS SHEATH”, and U.S. Patent Application Serial No. 63/867,193, filed Aug. 20, 2025, entitled “EXPANDABLE ACCESS SHEATH”, each of which are incorporated by reference herein in its entirety.

### TECHNICAL FIELD

The disclosure relates generally to medical devices and more particularly to medical devices that are adapted for use in percutaneous medical procedures.

### BACKGROUND

In some instances, performing percutaneous medical procedures may require the insertion and/or maneuvering of relatively large medical devices through a patient’s vasculature. Therefore, in some instances a relatively large vascular access sheath may be utilized to facilitate the insertion of relatively large medical devices into a patient’s vasculature. However, inserting a relatively large medical device into the vasculature through a large bore access sheath for an extended time period may, for example, lead to tissue damage (e.g., leg ischemia) due to a reduction in blood flow. Tissue damage resulting from placement of a relatively large medical device into the vasculature for an extended time period may be lessened by minimizing the size of the access sheath used to access the vessel. It may be desirable to configure a relatively large bore access sheath to have a reduced entry and/or crossing profile while also having robust column strength and kink resistance. Accordingly, it may be desirable to configure an access sheath to include an expandable distal end region having a reduced entry profile and a removable proximal portion having robust column strength and kink resistance. Example access sheaths including an expandable distal end region and a removeable proximal portion are disclosed herein.

### SUMMARY

This disclosure provides design, material, manufacturing method, and use alternatives for medical devices. An example access sheath for a medical device includes a hub attached to an elongate shaft, wherein the elongate shaft includes a proximal portion and an expandable portion. Further, the expandable portion is configured to shift between an unexpanded configuration and an expanded configuration. Further, the proximal portion includes a first diameter. Further, the expandable portion includes a second diameter in the unexpanded configuration, and wherein the second diameter is less than the first diameter.

Alternatively or additionally to any of the examples above, wherein expandable portion includes a third diameter in the expanded configuration, and wherein the third diameter is equal to the first diameter.

Alternatively or additionally to any of the examples above, wherein the expandable portion includes a distal end, a proximal end and a length extending between the distal end and the proximal end, and wherein at least a portion of the expandable portion tapers along the length.

Alternatively or additionally to any of the examples above, wherein the expandable portion includes an inner liner extending along a length of the expandable portion, the inner liner including at least one folded portion extending along the length of the expandable portion.

Alternatively or additionally to any of the examples above, wherein the at least one folded portion allows the inner liner to radially expand.

Alternatively or additionally to any of the examples above, wherein the at least one folded portion wraps along an inner surface of the inner liner.

Alternatively or additionally to any of the examples above, wherein the at least one folded portion includes a first folded pleat and a second folded pleat, wherein the first folded pleat and the second folded pleat extends along the length of the expandable portion.

Alternatively or additionally to any of the examples above, wherein the proximal portion includes a first frangible region and a second frangible region extending along a length of the proximal portion, and wherein the first frangible region is longitudinally aligned with the folded pleat, wherein the second frangible region is longitudinally aligned with the second folded pleat when the expandable portion is in the expanded configuration.

Alternatively or additionally to any of the examples above, wherein the proximal portion is configured to split along the first frangible region and the second frangible region.

Alternatively or additionally to any of the examples above, wherein the hub includes a first hub member configured to separate from a second hub member.

Alternatively or additionally to any of the examples above, wherein the separation of the first hub member from the second hub member initiates splitting the proximal portion along the first frangible region and the second frangible region.

Alternatively or additionally to any of the examples above, wherein splitting of the proximal portion permits the proximal portion to be removed from a medical device extending within a lumen of the proximal portion.

Alternatively or additionally to any of the examples above, wherein the first hub member includes a grip member extending away from the first hub member.

Alternatively or additionally to any of the examples above, wherein the expandable portion is configured to shift from the expanded configuration to a recovered configuration after the expandable portion has been expanded to the expanded configuration, and wherein the diameter of the expanded portion in the recovered configuration is less than the third diameter.

Another example access sheath for a medical device includes a hub attached to an elongate shaft, wherein the elongate shaft includes a proximal portion and an expandable portion. Further, the proximal portion includes a plurality of support members spaced from one another along a length of the proximal portion. Further, the expandable portion includes an inner liner having a plurality of folds disposed along an inner surface of the liner. Further, the expandable portion is configured to shift between a first unexpanded configuration and an expanded configuration. Further, the proximal portion includes a first diameter in a first configuration. Further, the expandable portion includes a second diameter in the unexpanded configuration, and wherein the second diameter is less than the first diameter.

The above summary is not intended to describe each disclosed embodiment or every implementation of the present disclosure. The Figures and Detailed Description, which follow, more particularly exemplify these examples.

### BRIEF DESCRIPTION OF DRAWINGS

FIG. 1 is a perspective view of an example medical device;

FIG. 2A is a perspective view of an example access sheath;

FIG. 2B is a perspective view of an example access sheath;

FIG. 3 illustrates a portion of the access sheath of FIG. 2;

FIG. 4 is a cross-sectional view of an example access sheath in a first configuration;

FIG. 5 is a cross-sectional view of the example access sheath of FIG. 4 in a second configuration;

FIG. 6A is a cross-sectional view of an example access sheath in a first configuration;

FIG. 6B is a cross-sectional view of the example access sheath of FIG. 6A in a second configuration;

FIG. 7 is a perspective view of an example cutter;

FIG. 8 is a perspective view of a blade assembly;

FIGS. 9-11 are cross-sectional views illustrating actuation of a cutter to cut a portion of the access sheath of FIG. 2;

FIG. 12 is a cross-sectional view illustrating an example cutter;

FIG. 13 is a cross-sectional view illustrating actuation of a cutter to cut a portion of the access sheath of FIG. 6;

FIG. 14 is a cross-sectional view taken along line 16-16 of the distal end region of the access sheath of FIGS. 2A-2B in a first, unexpanded configuration;

FIG. 15 is a cross-sectional view taken along line 16-16 of the distal end region of the access sheath of FIGS. 2A-2B in a second, partially-expanded configuration;

FIG. 16 is a cross-sectional view taken along line 16-16 of the distal end region of the access sheath of FIGS. 2A-2B in a third, expanded configuration;

FIG. 17 is a perspective view illustrating actuation of a cutter to cut the access sheath of FIG. 2;

FIG. 18 is an exploded view of the example access sheath of FIG. 2;

FIG. 19 is a perspective view illustrating the separation of the hub member and the splitting of the access sheath of FIG. 2B;

FIG. 20 is a cross-sectional view of the distal end region of an example access sheath of FIGS. 2A-2B in a first, unexpanded configuration;

FIG. 21 is a cross-sectional view of the distal end region of an example access sheath of FIGS. 2A-2B in a second, partially-expanded configuration;

FIG. 22 is a cross-sectional view of the distal end region of an example access sheath of FIGS. 2A-2B in a first, unexpanded configuration; and

FIG. 23 is a cross-sectional view of the distal end region of an example access sheath of FIGS. 2A-2B in a second, partially-expanded configuration.

While the disclosure is amenable to various modifications and alternative forms, specifics thereof have been shown by way of example in the drawings and will be described in detail. It should be understood, however, that the intention is not to limit the disclosure to the particular examples described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (e.g., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some examples”, “other examples”, etc., indicate that the embodiment described may include one or more particular features, structures, and/or characteristics. However, such recitations do not necessarily mean that all examples include the particular features, structures, and/or characteristics. Additionally, when particular features, structures, and/or characteristics are described in connection with one embodiment, it should be understood that such features, structures, and/or characteristics may also be used connection with other examples whether or not explicitly described unless clearly stated to the contrary.

The following detailed description should be read with reference to the drawings in which similar elements in different drawings are numbered the same. The drawings, which are not necessarily to scale, depict illustrative examples and are not intended to limit the scope of the disclosure.

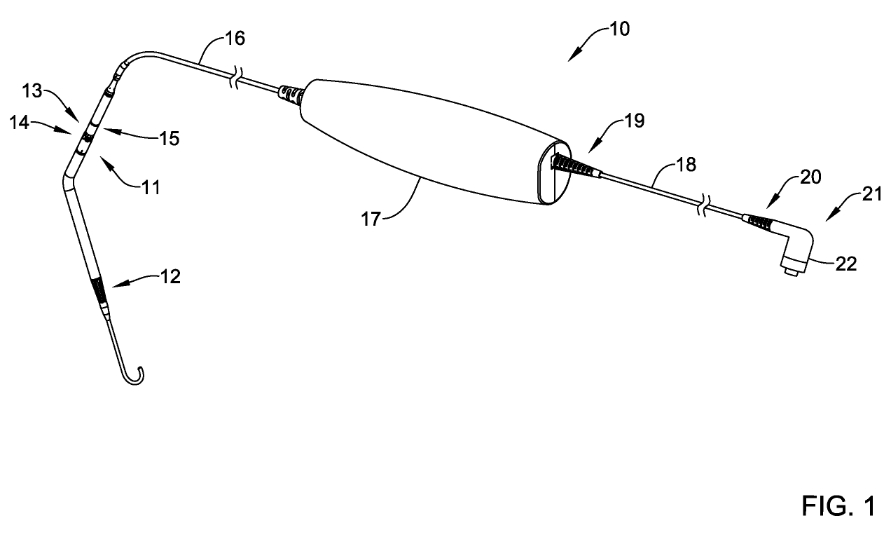

FIG. 1 shows an example percutaneous blood pump 10 (i.e., percutaneous cardiac assist device and/or circulatory support device) for connection to a medical device console (not shown). In the cases of blood pumps such as that depicted in FIG. 1, percutaneous cardiac assist devices and/or circulatory support devices support the pumping action of the heart. These devices may be positioned percutaneously into a patient’s heart, such as disposed across a valve opening such as, for example, an aortic valve. Such percutaneous blood pumps 10 assist the heart in pumping blood throughout the cardiovascular system, and the present disclosure provides uses, devices, and methods incorporating such devices as will be described herein. Such percutaneous blood pumps 10 may be connected to a medical device console for operating the blood pump and/or monitoring the operation of the blood pump and/or one or more patient parameters. However, a wide variety of medical devices are utilized to treat patients which are connected to a medical device console for operating the medical device and/or monitoring the operation of the medical device and/or one or more patient parameters. Accordingly, aspects of the present disclosure may be incorporated into a wide variety of medical devices.

Typically, a circulatory support device such as the example blood pump 10 shown in FIG. 1 includes a blood pump housing 11 including a blood inlet 12 at a blood inflow end of the blood pump housing 11 and a blood outlet 13 at a blood outflow end of the blood pump housing 11. The blood pump housing 11 may include an impeller 14 or other blood pumping device configured to convey blood through the blood pump housing 11 from the blood inlet 12 to the blood outlet 13. In some instances, the impeller 14 may be disposed within an impeller housing portion of the blood pump housing 11. The impeller 14 may be constructed of radially oriented blades protruding from a drive shaft and/or drive mechanism, for example. In some instances, the blood inlet 12 may be positioned and/or be positionable in a ventricle of the patient, while the blood outlet 13 may be positioned and/or be positionable in the aorta of the patient, such that the blood pump 10 pumps blood across the aortic valve. The impeller 14 may be driven by a motor 15 or other means of rotating the impeller 14 during operation thereof. In some instances, the motor 15 may be housed within a motor housing portion of the blood pump housing 11. In other instances, the motor 15 may be arranged at another location, if desired. For example, the motor 15 may be located on the blood pump 10 such that it remains outside the body when the blood pump 10 is in use. The motor 15 or other means of rotating the impeller 14 may be configured to rotate the impeller 14 through magnetism, electromagnetism, mechanical force, electrical force, and/or any combination or permutation of the aforementioned, such that the drive shaft and/or drive mechanism rotates the blades of the impeller 14, thereby creating a blood flow to propel blood through the blood pump housing 11 and beyond into the cardiovascular system. The blood pump 10 may include an elongate catheter shaft 16 extending proximally from the blood pump housing 11 for navigating the blood pump 10 to a desired location within the cardiovascular system of the patient. The catheter shaft 16 may extend proximally to a junction box 17, a handle, a manifold, or other structure of the blood pump 10 configured to remain exterior of the patient.

Furthermore, the blood pump 10 may include a cable 18 at a proximal end thereof having a connection interface 21 (e.g., connector) at an end thereof. The cable 18 may have a first, distal end 19 connected to the junction box 17, a handle, a manifold, or other structure of the blood pump 10, and a second, proximal end 20 having the connection interface 21 including a connector 22 that is configured to be connected to a medical device console (e.g., blood pump console). The cable 18 may include one or more of, an electrical pathway for powering the motor 15, an electrical pathway for powering one or more sensors, an electrical pathway for transmitting signals from one or more sensors associated with the blood pump 10, a fiber optic filament for transmitting signals from one or more sensors associated with the blood pump 10, or other communication conduits. In some instances, the cable 18 and associated connector 22, may be considered a single-connector cable device providing the only connection between the blood pump 10 and a medical device console controlling the blood pump 10.

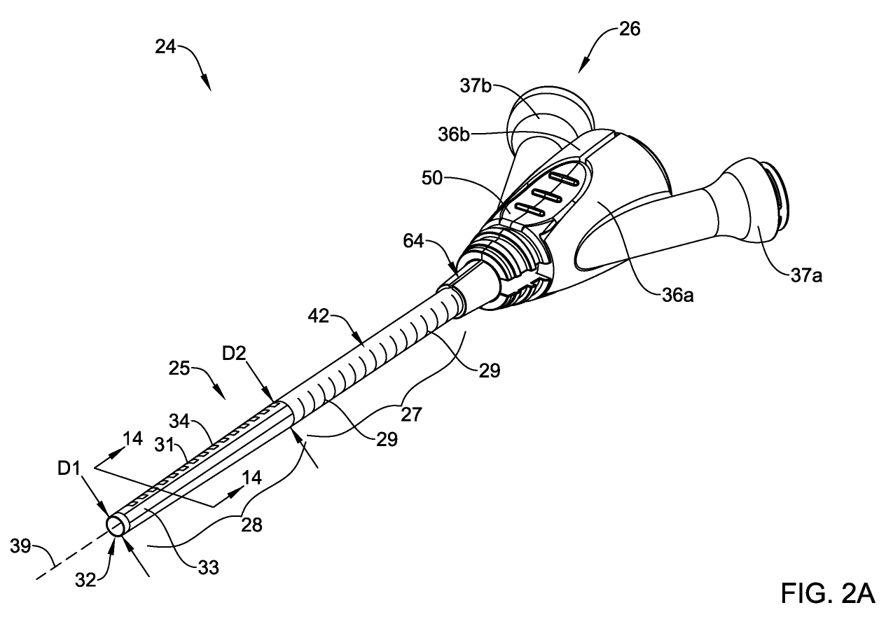

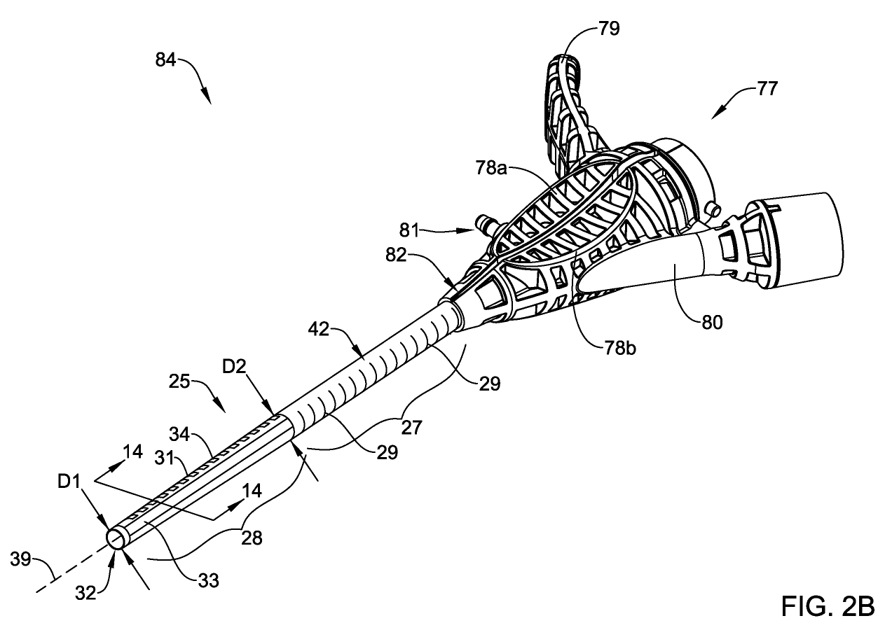

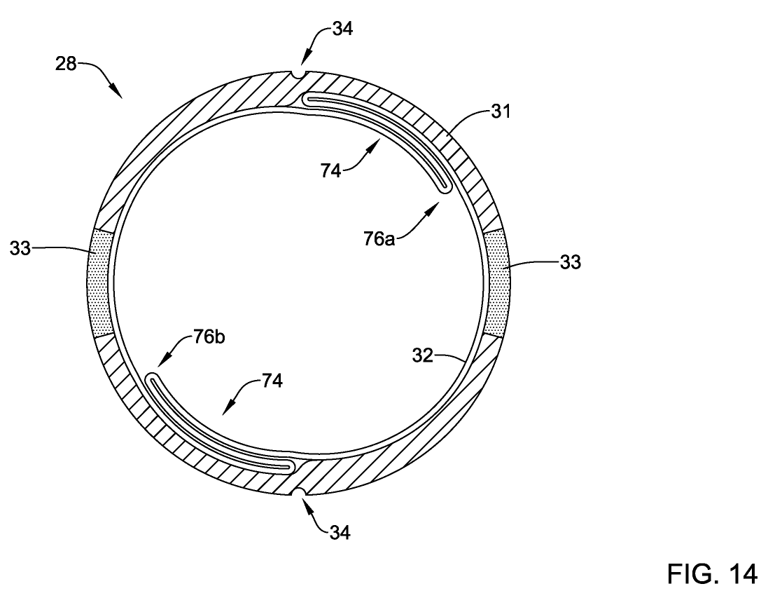

FIG. 2A is a perspective view of an example vascular access sheath 24 for use in providing vascular access for introducing a medical device (e.g., the percutaneous blood pump of FIG. 1) into a vasculature at a vascular access site. The access sheath 24 may include an elongate shaft 25 having a proximal portion 27 and a distal, expandable portion 28. The proximal portion 27 may be referred to as a non-expandable portion. As described herein, the proximal portion 27 may include a portion of the access sheath 24 that is cuttable, splitable, peelable, separable, breakable, etc. along a frangible region 42. As will be described in greater detail herein, the proximal portion 27 may include one or more reinforcing members 29 positioned along the length of the proximal portion 27. Additionally, FIG. 2A illustrates that the expandable portion 28 may include an elongated compliant liner 32 (e.g., tubular membrane, etc.) having a lumen extending therein. The expandable portion 28 may also include an outer sheath 31 extending along (e.g., covering) the liner 32. FIG. 2A further illustrates that the expandable portion 28 may include one or more support members 33 (e.g., spines) extending along the length of the outer sheath 31. The arrangement of the liner 32, the outer sheath 31 and support members 33 of the expandable portion 28 is described in greater detail with respect to FIGS. 14-16.

Further, FIG. 2A illustrates that the outer sheath 31 may include one or more apertures (e.g., openings) 34 extending through at least a portion of the wall of outer sheath 31. As shown in FIG. 2A, the one or more apertures 34 may be longitudinally aligned with one another along the central longitudinal axis 39 of the expandable portion 28. Additionally, as shown in FIGS. 14-16, the apertures 34 may be positioned between adjacent support members 33.

FIG. 2A illustrates that the distal end of the expandable portion 28 may have a diameter D₁in a pre-expanded configuration. In some examples, diameter D₁ may be about 0.100 inches (2.54 millimeters) to about 0.150 inches (3.81 millimeters). Further, FIG. 2A illustrates that, in the pre-expanded configuration shown in FIG. 2A, the expandable portion may taper along its length from the diameter D₁ at the distal end of the expandable portion 28 to a diameter D₂ at the proximal end of the expandable portion 28. In some examples, diameter D₂ may be about 0.200 inches (5.08 millimeters) to about 0.25 inches (6.35 millimeters). It can be appreciated that the diameter D₁ may provide a reduced crossing profile which helps to minimize vascular damage upon insertion of the access sheath into a body lumen.

FIG. 2A further illustrates that the access sheath 24 may include a hub 26 coupled to the proximal end of the portion 27. As will be described in greater detail herein, the access sheath 24 may also include a removeable cutter 50 coupled to the hub 26.

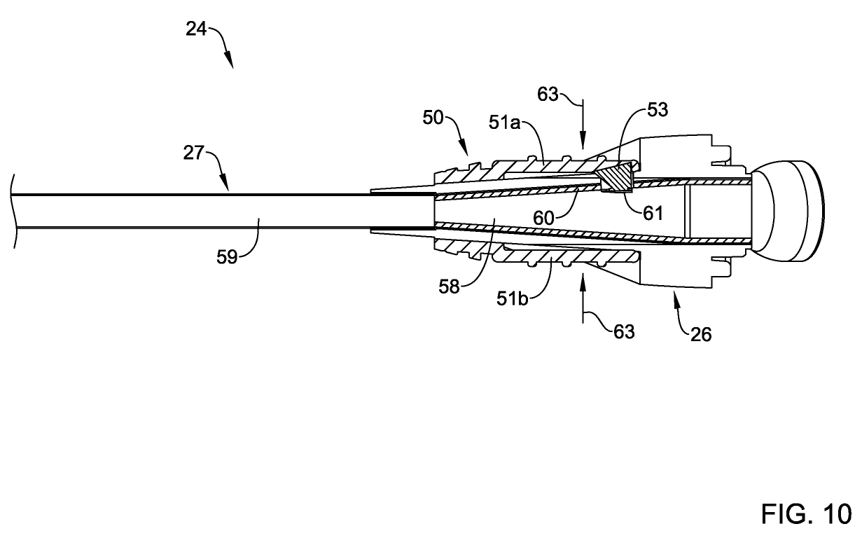

The splitable hub 26 may include a first hub member 36a and a second hub member 36b releasably attached together. In some examples, the first hub member 36a and the second hub member 36b may be releasably attached to one another via a splitable membrane 60 (shown in FIG. 10). In some examples, the splitable membrane 60 attaching the first hub member 36a and the second hub member 36b may be formed from a soft durometer material. The hub 26 may also include a first side port 37a and a second side port 37b. In some examples, the hub 26 may only include the first side port 37a. In some examples, the first side port 37a and a second side port 37b may be configured to permit a user to grasp each of the first side port 37a and a second side port 37b to separate the first hub member 36a and the second hub member 36b. In some instances, the first side port 37a may extend at an acute angle from the first hub member 36a and the second side portion 37b may extend at an acute angle from the second hub member 36b. The first side port 37a and the second side port 37b may provide access to one or more lumens extending through the elongate shaft 25.

FIG. 2B is a perspective view of another example vascular access sheath 84 for use in providing vascular access for introducing a medical device (e.g., the percutaneous blood pump of FIG. 1) into a vasculature at a vascular access site. The access sheath 84 may be similar in form and function to the access sheath 24 described herein, and thus discussion of aspects, features, functionality, and components thereof, may be similarly attributed to the access sheath 84. For example, the access sheath 84 may include an elongate shaft 25 having a proximal portion 27 and a distal, expandable portion 28. Like that described with respect to the access sheath 24, the proximal portion 27 may include a portion of the access sheath 24 that is cuttable, splitable, peelable, separable, breakable, etc. along a frangible region 42. Further, the proximal portion 27 may include one or more reinforcing members 29 positioned along the length of the proximal portion 27. Additionally, FIG. 2B illustrates that the expandable portion 28 may include an elongated compliant liner 32 (e.g., tubular membrane, etc.) having a lumen extending therein. The expandable portion 28 may also include an outer sheath 31 extending along (e.g., covering) the liner 32. FIG. 2B further illustrates that the expandable portion 28 may include one or more support members 33 (e.g., spines) extending along the length of the outer sheath 31. Further, FIG. 2B illustrates that the outer sheath 31 may include one or more apertures (e.g., openings) 34 extending through at least a portion of the wall of outer sheath 31. As shown in FIG. 2B, the one or more apertures 34 may be longitudinally aligned with one another along the central longitudinal axis 39 of the expandable portion 28.

FIG. 2B illustrates that the distal end of the expandable portion 28 may have a diameter D₁in a pre-expanded configuration. In some examples, diameter D₁ may be about 0.100 inches (2.54 millimeters) to about 0.150 inches (3.81 millimeters). Further, FIG. 2B illustrates that, in the pre-expanded configuration shown in FIG. 2B, the expandable portion may taper along its length from the diameter D₁ at the distal end of the expandable portion 28 to a diameter D₂ at the proximal end of the expandable portion 28. In some examples, diameter D₂ may be about 0.200 inches (5.08 millimeters) to about 0.25 inches (6.35 millimeters). It can be appreciated that the diameter D₁ may provide a reduced crossing profile which helps to minimize vascular damage upon insertion of the access sheath into a body lumen.

FIG. 2B further illustrates that the access sheath 84 may include a hub 77 coupled to the proximal end of the portion 27. The splitable hub 77 may include a first hub member 78a and a second hub member 78b releasably attached together. In some examples, the first hub member 78a and the second hub member 78b may be releasably attached to one another via a splitable membrane 60 (shown in FIG. 10). In some examples, the splitable membrane 60 attaching the first hub member 78a and the second hub member 78b may be formed from a soft durometer material. The hub 77 may also include a grip member 79 and a side port 80. In some examples, the grip member 79 and/or the side port 80 may be configured to permit a user to grasp each of the grip member 79 and/or the side port 80 to separate the first hub member 78a and the second hub member 78b. In some instances, the grip member 79 may extend at an acute angle from the first hub member 78a and the side port 80 may extend at an acute angle from the second hub member 78b. The side port 80 may provide access to one or more lumens extending through the elongate shaft 25.

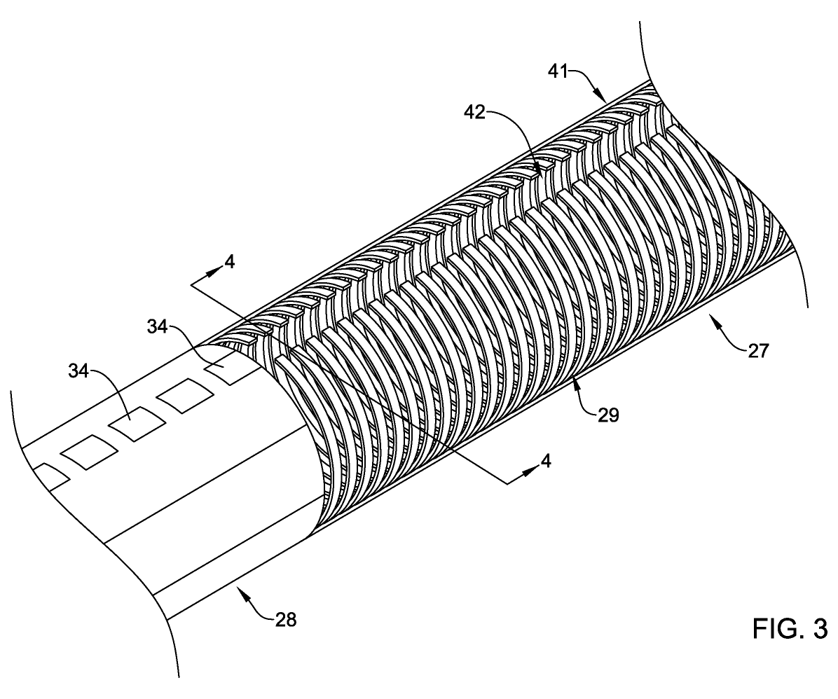

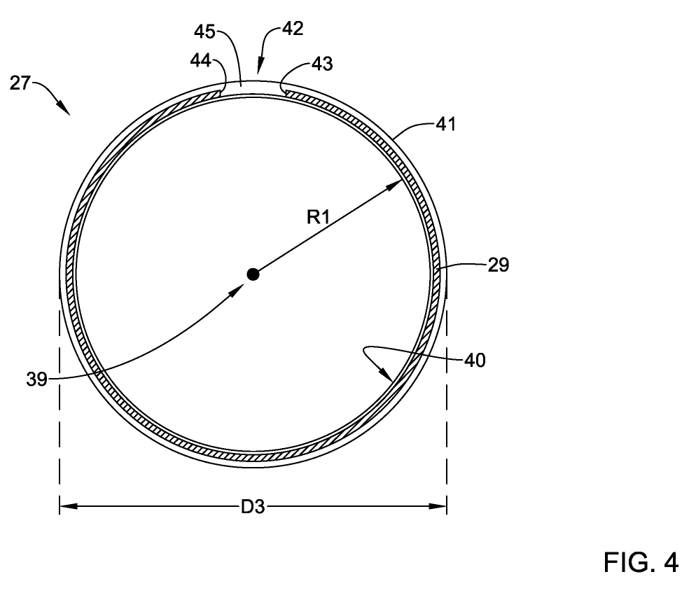

FIG. 3 illustrates the distal end region of the proximal portion 27 and the proximal end region of the expandable portion 28 of the elongate shaft 25 of either the access sheath 24 of FIG. 2A or the access sheath 84 of FIG. 2B. As described herein, FIG. 3 illustrates that the elongate shaft 25 of the access sheath 24/84 may include a plurality of reinforcing members 29 aligned along the length of the proximal portion 27. It can be appreciated from FIG. 3 that each respective reinforcing member 29 may be positioned adjacent to one another along the longitudinal axis 39 (shown in FIGS. 2A and 2B) of the elongate shaft 25. FIG. 3 further illustrates that each of the plurality of reinforcing members 29 may partially extend about the longitudinal axis 39 of the elongate shaft 25. For example, FIG. 3 illustrates that each of the respective reinforcing members 29 may extend only partially around the longitudinal axis 39 of the elongate shaft 25, whereby each of the plurality of reinforcing members 29 includes a first end circumferentially spaced away from a second end. In some examples, the portion of the proximal portion 27 positioned between the first end and the second end of each reinforcing member 29 may define a frangible region 42 along the elongate shaft 25. As will be described in greater detail below, the frangible region 42 may include the region along the proximal portion 27 in which the cutter 50 may cut through the wall 45 (shown in FIG. 4) of the proximal portion 27 and/or the frangible region 42 may include the region along the proximal portion 27 in which the elongate shaft 25 of the access sheath 84 is split or otherwise separated.

FIG. 3 further illustrates the plurality of apertures 34 longitudinally aligned with one another along the proximal end region of the expandable portion 28. It can be appreciated from FIG. 3 that the plurality of apertures 34 may be longitudinally aligned with the frangible region 42 of the proximal portion 27.

FIG. 4 illustrates an example cross-sectional view of the proximal portion 27 taken along line 4-4 of FIG. 3. FIG. 4 illustrates the proximal portion 27 in a first configuration. FIG. 4 illustrates that the distal end of the proximal portion 27 may have a diameter D₃in the first, pre-expanded configuration shown in FIG. 3. In some instances, the diameter D₃ may be equal to the diameter D₂ of the expandable portion 28 in the pre-expanded configuration, the diameter D₃ may be substantially equivalent to the diameter D₂ of the expandable portion 28 in the pre-expanded configuration, or the diameter D₃ may be greater than the diameter D₂ of the expandable portion 28 in the pre-expanded configuration. In some examples, the diameter D₃ may be about 0.200 inches (5.08 millimeters) to about 0.25 inches (6.35 millimeters).

FIG. 4 illustrates that the proximal portion 27 may further include a lubricious liner 40 (shown in FIG. 4) extending along the length of the proximal portion 27. It can be appreciated that the lubricious liner 40 may be positioned radially inward of the plurality of the reinforcing members 29. It can be further appreciated that, in some examples, an outer surface of the lubricious liner 40 may contact and be attached to the inner surface of each of the plurality of reinforcing members 29.

Additionally, the access sheath 24 may also include an outer layer 41. The outer layer 41 may extend along (e.g., cover) both the plurality of reinforcing members 29 and the lubricious liner 40. FIG. 4 further illustrates that the outer layer 41 may extend continuously around the longitudinal axis 39 of the elongate shaft 25. As will be discussed in greater detail below, the plurality of reinforcing members 29 may be positioned between (e.g., sandwiched between) the lubricious liner 40 and the outer layer 41. In some examples, the outer layer 41 may be attached to both the lubricious liner 40 and the plurality of reinforcing members 29. FIG. 4 further illustrates that the proximal portion 27 may include a radius R₁ when in the first configuration.

Additionally, FIG. 4 illustrates that the reinforcing member 29 may extend partially around the longitudinal axis 39 of the proximal portion 27 of the elongate shaft 25. For example, FIG. 4 illustrates that the reinforcing member 29 may be configured such that a first end 43 of the reinforcing member 29 is spaced away from a second end 44 of the reinforcing member 29. As discussed herein, FIG. 4 also illustrates the frangible region 42 positioned between the first end 43 and the second end 44 of the reinforcing member 29.

As discussed herein, the elongate shaft 25 may be configured to permit the cutter to cut through a wall 45 of the elongate shaft 25 along the frangible region 42. For example, the access sheath 24 described herein may be utilized to insert and position a relatively large medical device (e.g., blood pump 10) within a patient. However, after inserting and positioning the large medical device (e.g., blood pump 10), it may be desirable to remove the access sheath 24 from the medical device (e.g., remove it from the shaft 16 of the blood pump 10 which may be extending through the access sheath 24). Accordingly, a user may utilize the cutter 38 (shown in FIG. 2A) to cut through the wall 45 of the proximal portion 27 of the access sheath 24, thereby purposefully splitting the wall 45 of the proximal portion 27 along the frangible region 42.

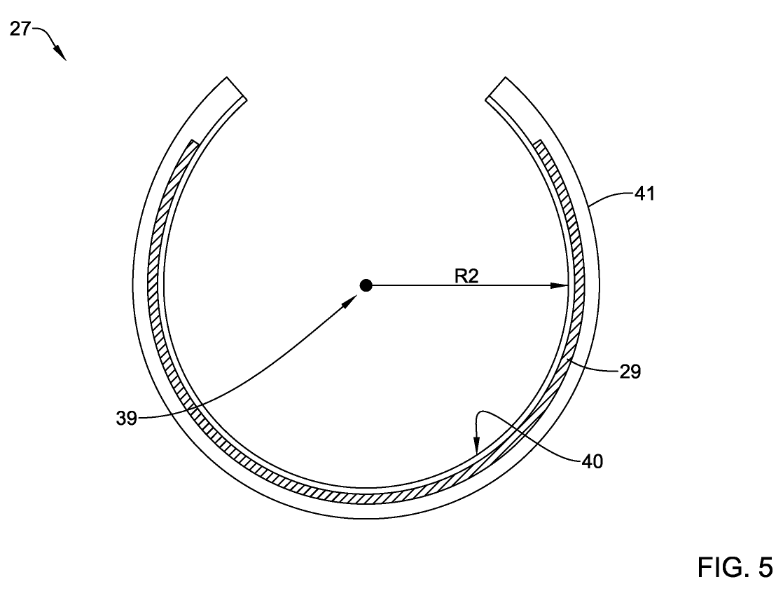

Further, in some examples, each of the reinforcing members 29 of the access sheath 24/84 may be configured to be positioned between the lubricious liner 40 and the outer layer 41 (such as that shown in FIG. 4) under circumferential tension. In other words, during the manufacturing process, the reinforcing members 29 as shown in FIG. 4 may be placed under circumferential tension while the lubricious liner 40 and the outer layer 41 are formed around each respective reinforcing member 29, thereby maintaining each reinforcing member 29 under circumferential tension until the cutter 50 is utilized to cut the wall of the proximal portion 27. It can be appreciated that the term circumferential tension, as used herein, may describe a first configuration in which the reinforcing member 29 is held under tension in a first position (as shown in FIG. 4) and whereby each reinforcing member 29 may be biased to expand radially outward to a second position (as shown in FIG. 5) when the proximal portion 27 is cut, thereby releasing each reinforcing member 29 from the first configuration and allowing each reinforcing member 29 to expand radially outward to the second position shown in FIG. 5. FIG. 5 further illustrates that the proximal portion 27 may include a radius R₂ in the second configuration which is greater than the radius R₁ of the proximal portion 27 shown in the first configuration of FIG. 4. In some examples, each reinforcing member 29 may be described as having an outward radial self-bias defined by the configuration illustrated in FIG. 5.

In other examples, however, the reinforcing members 29 may not be placed under circumferential tension during manufacturing. In such examples, the reinforcing members 29 may be positioned between the lubricious liner 40 and the outer layer 41 without being biased to separate from one another, which may permit the proximal portion 27 to maintain a substantially circular cross-sectional shape and reduce or prevent ovalization of the proximal portion 27 during manufacturing, for example.

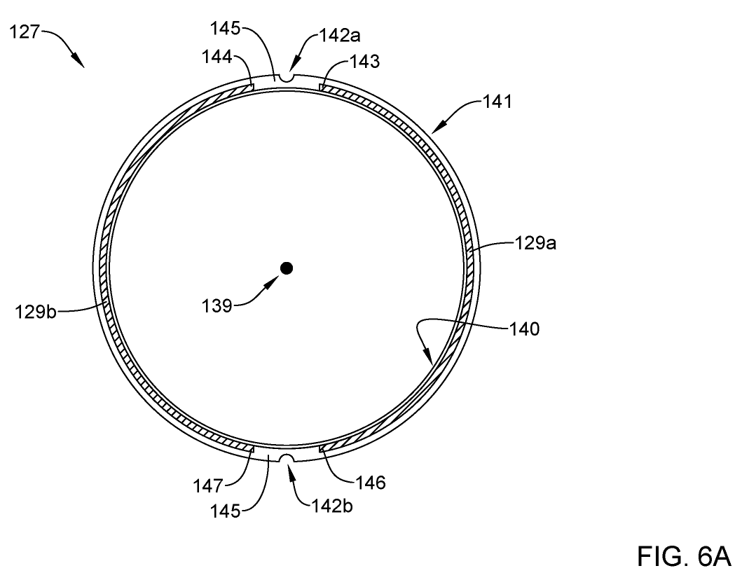

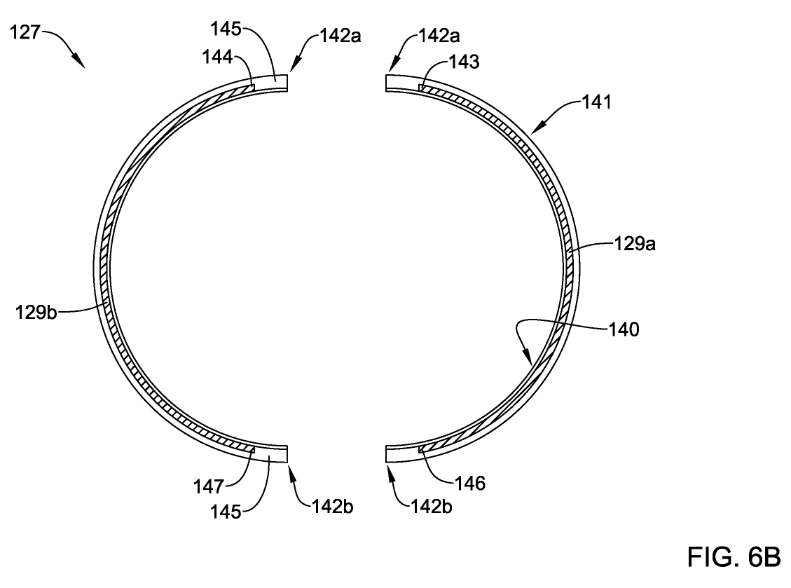

FIG. 6A illustrates an example cross-sectional view of another example proximal portion 127 of the elongate shaft 25 (shown in FIGS. 2A-2B). The proximal portion 127 may be similar in form and function to the proximal portion 27 of the access sheaths 24/84 described herein. For example, FIG. 6A illustrates that the proximal portion 127 may include both a first reinforcing member 129a and a second reinforcing member 129b positioned between (e.g., sandwiched between) the inner lubricious liner 140 and the outer layer 141. As illustrated in FIG. 6A, the first reinforcing member 129a and the second reinforcing member 129b may be circumferentially offset from one another. FIG. 6A illustrates that the inner lubricious liner 140 may extend continuously around the longitudinal axis 139 of the proximal portion 127.

Additionally, FIG. 6A illustrates that each of the reinforcing members 129a, 129b extend partially around the longitudinal axis 139 of the proximal portion 127. For example, FIG. 6A illustrates that the first reinforcing member 129a and the second reinforcing member 129b are configured such that a first end 143 of the first reinforcing member 129a is spaced away from the first end 144 of the second reinforcing member 129b and a second end 146 of the first reinforcing member 129a is spaced away from a second end 147 of the second reinforcing member 129b.

FIG. 6A illustrates that the proximal portion 127 may include a first frangible region 142a positioned between the first end 143 of the first reinforcing member 129a and the first end 144 of the second reinforcing member 129b. Additionally, FIG. 6A illustrates that the proximal portion 127 may include a second frangible region 142b positioned between the second end 146 of the first reinforcing member 129a and the second end 147 of the second reinforcing member 129b. FIG. 6A illustrates that each of the first frangible region 142a and the second frangible region 142b may include one or more regions (e.g., score line, weakened area, groove, perforations, etc.) which extend radially inward from an outer surface of the outer layer 141. In some examples, the frangible region 142a may include a series of indentations (e.g., perforations) which may be aligned with one another along the central longitudinal axis 139 of the proximal portion 127. Similarly, the frangible region 142b may include a series of indentations which may be aligned with one another along the central longitudinal axis 139 of the proximal portion 127. In some examples, the frangible regions 142a, 142b may each include perforated frangible regions whereby each frangible region 142a, 142b includes a plurality of perforations extending longitudinally along the outer layer 141.

However, in other examples, the frangible regions 142a, 142b of the proximal portion 127 may not include indentations or perforations. Instead, the frangible regions 142a, 142b may be defined by a region of one or more of the outer layer 141and the inner lubricious liner 140 having a reduced wall thickness and/or a reduced outer diameter relative to adjacent portions of the proximal portion 127, thereby defining a region (e.g., score line, weakened area, groove, etc.) configured to preferentially separate when a threshold pull-away force is applied.

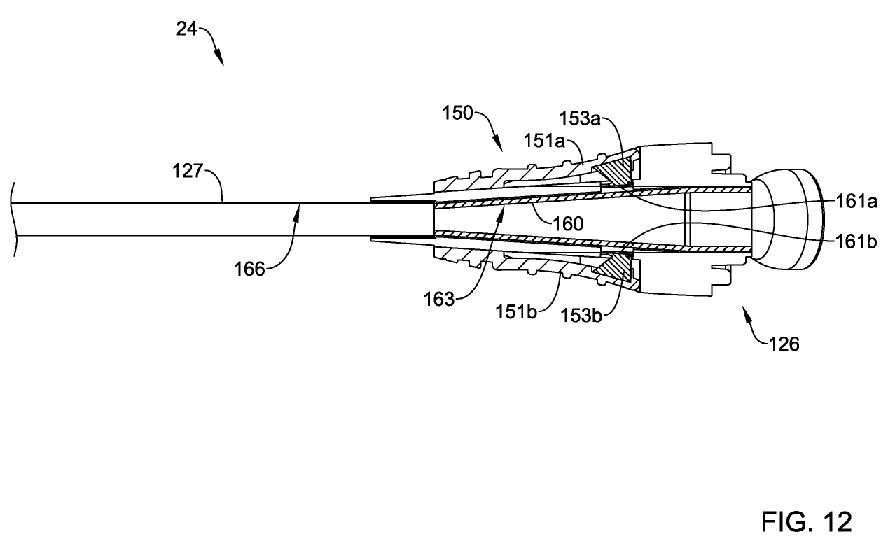

Similar to that described with respect to the embodiment illustrated in FIG. 4, in some examples, the proximal portion 127 may be configured to permit a first blade of the access sheath 24 (shown in FIG. 2A) to cut through the wall 145 of the proximal portion 127 along the first frangible region 142a and a second blade to cut through the wall 145 of the proximal portion 127 along the second frangible region 142b. For example, a user may utilize the cutter 150 (shown in FIG. 12) to cut through the wall 145 of the first frangible region 142a and the second frangible region 142b of the proximal portion 127, thereby purposefully splitting the wall 145 of the proximal portion 127 along the first frangible region 142a and the second frangible region 142b. Further, in some examples, each of the reinforcing members 129a, 129b may be configured to be positioned between the lubricious liner 140 and the outer layer 141 under circumferential tension. In other words, during the manufacturing process, the reinforcing members 129a, 129b as shown in FIG. 6A may be placed under circumferential tension while the liner 140 and the outer layer 141 are formed around each respective reinforcing member 129a, 129b.

In other examples, however, the reinforcing members 129a, 129b may not be placed under circumferential tension during manufacturing. In such examples, the reinforcing members 129a, 129b may be positioned between the lubricious liner 140 and the outer layer 141 without being biased to separate from one another, which may permit the proximal portion 127 to maintain a substantially circular cross-sectional shape and reduce or prevent ovalization of the proximal portion 127 during manufacturing, for example.

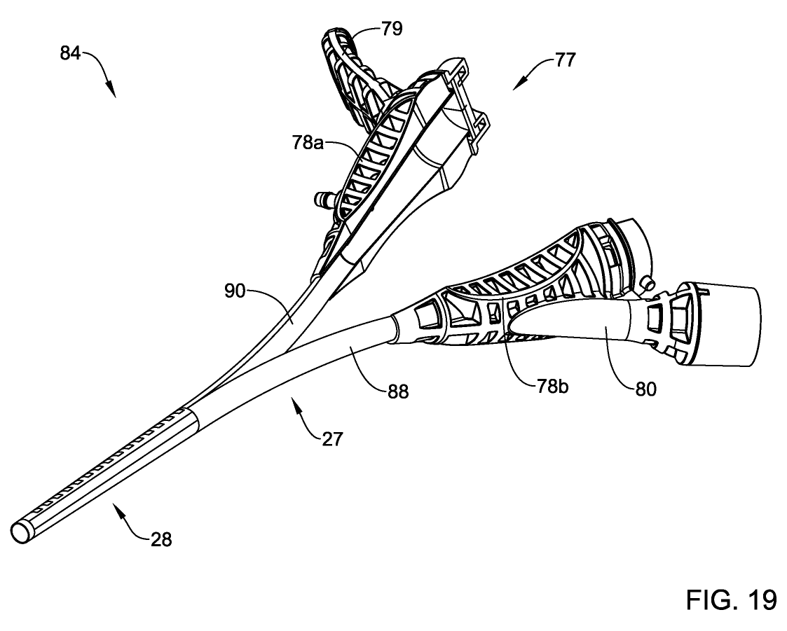

Further, FIG. 6B illustrates that, in some examples, the access sheath 84 (shown in FIG. 2B) may be configured such that the proximal portion 127 may be split along either the first frangible region 142a or the second frangible region 142b, or split along both the first frangible region 142a and the second frangible region 142b. For example, as described with respect to FIG. 2B, the hub 77 (shown in FIG. 2B) may be configured to split into the first hub member 78a and a second hub member 78b, whereby each of the first hub member 78a and a second hub member 78b may be gripped by a user and utilized to peel the proximal portion 127 along both the first frangible region 142a and the second frangible region 142b. Splitting the hub 77 into the first hub member 78a and a second hub member 78b and peeling the proximal portion 127 along both the first frangible region 142a and the second frangible region 142b is further illustrated and described with respect to FIG. 19.

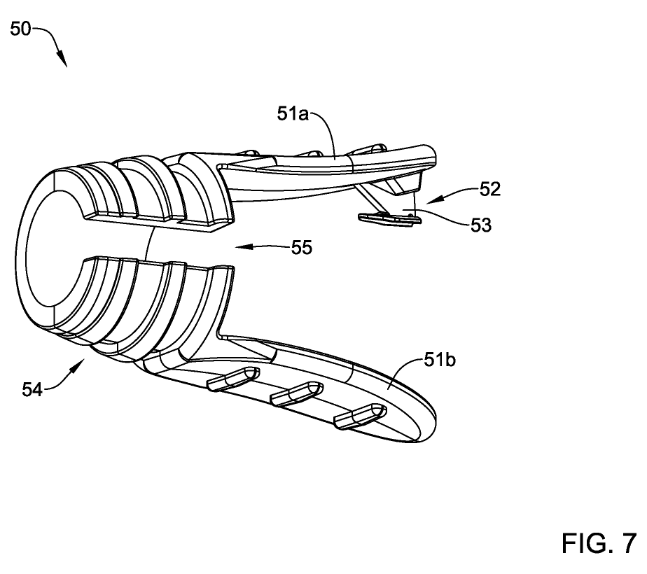

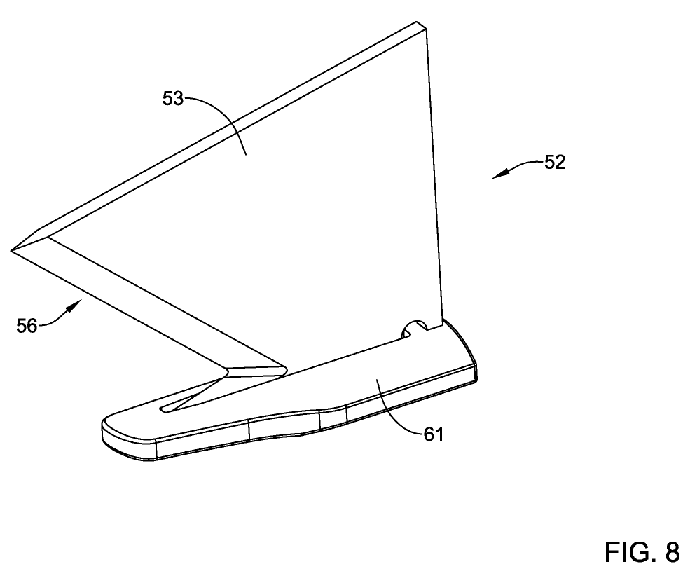

FIG. 7 illustrates a cutter 50 of the access sheath 24 including a blade assembly 52 (also shown in FIG. 8). FIG. 7 illustrates that the cutter 50 may include a body portion 54. Further, the body portion 54 may include a longitudinally-extending opening 55 extending through a wall of the body portion 54. Accordingly, the body portion 54 may be generally “C-shaped” when the cutter 50 is viewed from the distal end. As will be discussed in greater detail below, the opening 55 may be utilized to remove the cutter 50 from another medical device (e.g., guidewire, etc.) used in conjunction with the access sheath 24 during a medical procedure.

FIG. 7 further illustrates that the cutter 50 may further include a first grip member 51a and a second grip member 51b, whereby each grip member 51a, 51b may extend away from the body portion 54 in a proximal direction. It can be appreciated that the cutter 50 may be configured such that a user may grasp the grip members 51a, 51b with their fingers to manipulate the cutter 50. It can be appreciated that, in some examples, each grip member 51a, 51b may include one or more ergonomic features to aid a user in grasping and manipulating the cutter 50.

FIG. 7 further illustrates that the cutter 50 may further include a cutting assembly 52 attached to an inner facing surface (e.g., underside) of the grip member 51a. FIGS. 7-8 illustrate that the cutting assembly 52 may include a cutting blade 53 having a distally facing cutting surface 56. FIG. 7 illustrates that the cutting blade 53 may extend vertically downward from the underside of the first grip member 51a, thereby positioning the cutting surface 56 such that it faces toward the elongate shaft 25. FIG. 8 further illustrates that the cutting assembly 52 may further include a guide member 61 coupled to the cutting blade 53.

While FIG. 7 illustrates the cutting assembly 52 attached to the first grip member 51a, it can be appreciated that, in some examples, the cutting assembly 52 may be attached to the second grip member 51b. In yet other examples (such as that shown in FIG. 10), the cutter 50 may be configured to include a first cutting assembly 52 attached to the first grip member 51a and a second cutting assembly attached to the second grip member 51b. It can be appreciated that the first grip member 51a may include a top surface facing away from the longitudinal axis 39 of the access sheath 24 and a bottom surface facing toward the longitudinal axis 39 of the access sheath 24. Additionally, the second grip member 51b may include a top surface facing away from the longitudinal axis 39 of the access sheath 24 and a bottom surface facing toward the longitudinal axis 39 of the access sheath 24.

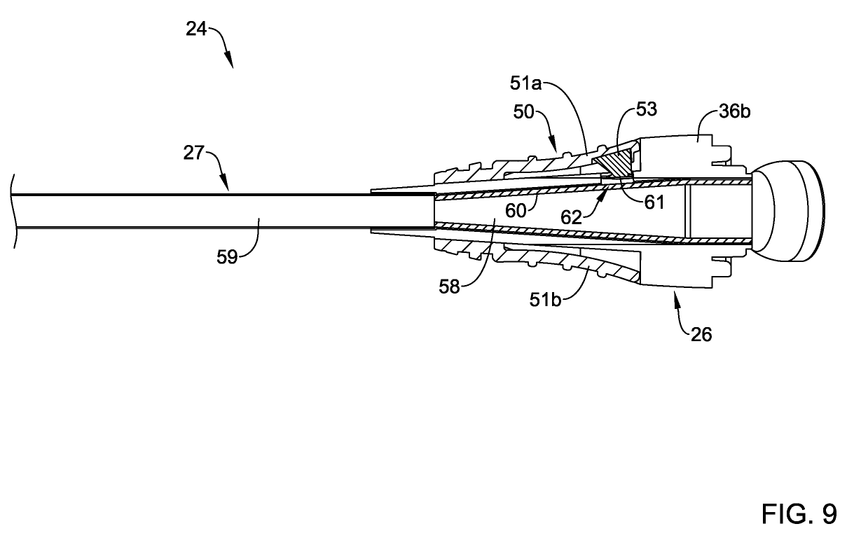

FIG. 9 illustrates a cross-sectional view of a portion of the access sheath 24 including the cutter 50 removably coupled to the second hub member 36b of the hub 26. It can be appreciated from FIG. 9 that the hub 26 may include an inner lumen 58 which is in contact with an inner lumen 59 of the proximal portion 27. FIG. 9 further illustrates the first grip member 51a and the second grip member 51b in a pre-cutting configuration, whereby neither the first grip member 51a and the second grip member 51b have been actuated to initiate translation of the cutter 50 along either the hub 26 or proximal portion 27. In some examples, the first hub member 36a and the second hub member 36b may be releasably attached to one another via a splitable membrane 60. In some examples, the splitable membrane 60 attaching the first hub member 36a and the second hub member 36b may be positioned along an inner surface of the first hub member 36a and the second hub member 36b of the hub 26. In some examples, the splitable membrane 60 attaching the first hub member 36a and the second hub member 36b may be formed from a soft durometer material.

FIG. 9 further illustrates that the guide member 61 of the cutting assembly 52 may be aligned with a vertical aperture 62 (also shown in FIG. 14) positioned within the hub 26 (within an inner chamber of the hub 26). The vertical aperture 62 may be formed by a combination of recesses in both the first hub member 36a (shown in FIG. 2A) and the second hub member 36b. It can be appreciated that the surfaces of the first hub member 36a (shown in FIG. 2A) and the second hub member 36b which form the vertical aperture 62 may prevent the cutter 50 from translating in a proximal-to-distal direction as the distal region of the guide member 61 may contact the surfaces of the first hub member 36a (shown in FIG. 2A) and the second hub member 36b which form the vertical aperture 62.

FIG. 10 illustrates the actuation (e.g., pinching, squeezing, etc.) of the first grip member 51a and the second grip member 51b relative to the hub 26. The actuation of the first grip member 51a and the second grip member 51b is illustrated by the directional arrows 63 in FIG. 10. As discussed herein, it can be appreciated that a user may use both of their hands to actuate the cutter 50 relative to the hub 26. For example, a user may grasp and pinch the first grip member 51a and the second grip member 51b of the cutter 50 with the fingers of one hand while holding onto the hub 26 with the fingers of their other hand. FIG. 10 further illustrates that the pinching of the first grip member 51a and the second grip member 51b may translate the cutting assembly 52 downward within the aperture of the 62 (shown in FIG. 9), whereby the downward translation of the cutting assembly 52 (shown in FIGS. 7-8) punctures the splitable membrane 60. In other words, the downward translation of the cutting assembly 52 drives the first grip member 51a through the splitable membrane 60, thereby shifting the guide member 61 from a position within the aperture 62 to a position within the lumen 58 of the hub 26, thereby freeing the guide member 61 from within the aperture 62.

Additionally, because the blade 53 may be sized such that its width may permit it to fit within a longitudinally-extending channel 64 (shown in FIG. 2A) of the hub 26. Accordingly, after the guide member 61 is translated downward and freed from the aperture 62 (via puncturing the splitable membrane 60), the cutter 50 may be free to translate in a proximal-to-distal direction within the hub 26. It can be appreciated that once the cutter 50 begins the proximal-to-distal translation within the hub 26, a top surface of the guide member 61 may contact an inner surface splitable membrane 60, thereby preventing the first grip member 51a and the second grip member 51b from expanding radially outward and pulling the blade 53 out of the channel 64 (shown in FIG. 2A).

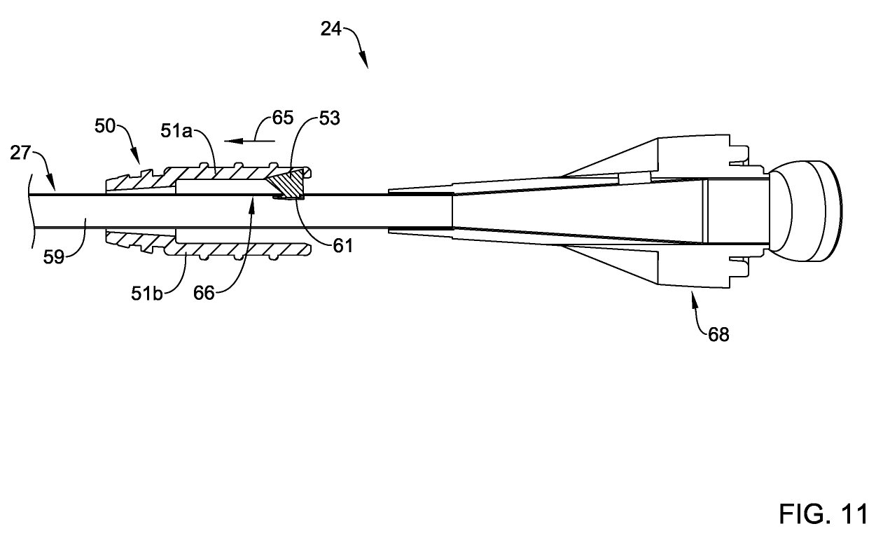

FIG. 11 illustrates the cutter 50 being translated in a proximal-to-distal direction to initiate cutting of the proximal portion 27 along the frangible region 42 (shown in FIGS. 2A, 3-4). Translation of the cutter 50 in a proximal-to-distal direction is depicted by the directional arrow 65 in FIG. 11. FIG. 11 illustrates that translation of the cutter 50 in a proximal-to-distal direction may permit the cutting surface 56 (shown in FIG. 8) of the blade 53 to engage and cut the wall of the proximal portion 27. Additionally, it can be appreciated from FIG. 11 that the access sheath 24 may be configured such the inner surface 60 (shown in FIG. 10) of the hub 68 (whereby the hub 68 may represent any hub disclosed herein, including the hub 26 disclosed herein) is circumferentially aligned with an inner surface 66 of the proximal portion 27. Accordingly, as the cutter 50 is translated in a proximal-to-distal direction, the top surface of the guide member 61 may contact the inner surface 66 of the proximal portion 27, thereby preventing the first grip member 51a and the second grip member 51b from expanding radially outward and pulling the blade 53 out of the wall of the proximal portion 27 and thereby allowing the cutting surface 56 of the blade 53 to cut the proximal portion 27.

FIG. 12 illustrates a cross-sectional view of an alternative embodiment of cutter 50 described herein. For example, FIG. 12 illustrates a cutter 150 positioned along a hub 126, whereby the cutter 150 and the hub 126 may be similar in form and function to the cutter 50 and hub 26 described herein. FIG. 12 illustrates that, in some examples, the cutter 150 may include a first blade 153a and a first guide member 161a coupled to the first grip member 151a and a second blade 153b and a second guide member 161b coupled to the second grip member 151b. The first blade 153a and the second blade 153b may be similar in form and function to the blade 53 described herein. The first guide member 161a and the second guide member 161b may be similar in form and function to the guide member 61 described herein. FIG. 12 illustrates that the first blade 153a may be circumferentially offset from the second blade 153b. FIG. 12 further illustrates that the hub may include a splitable membrane 160 which may be similar in form and function to the splitable membrane 60 described herein.

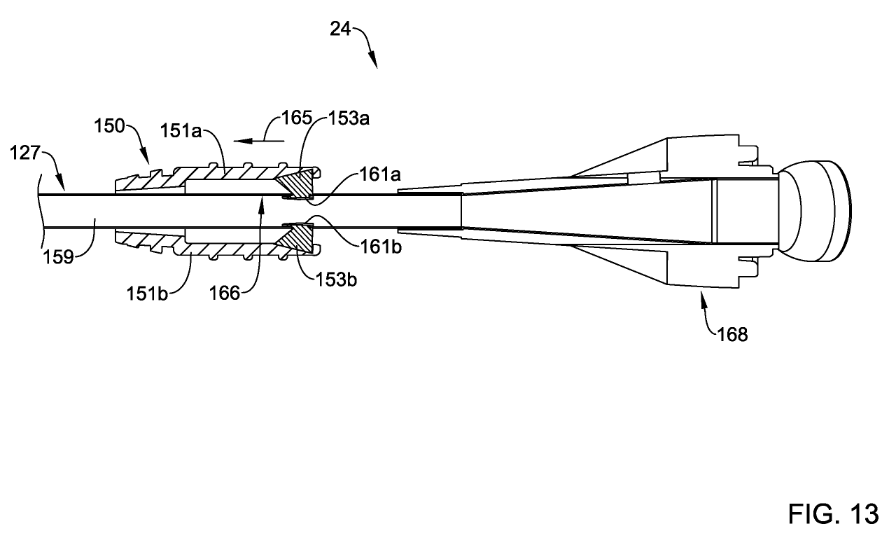

Referring to FIGS. 6A and 13, it can be appreciated that when actuated (similar to the actuation of the single blade cutter embodiment shown in FIGS. 7-11), the first blade 153a may be configured to cut the proximal portion 127 (shown in FIG. 6A) along the cutting region 142a (shown in FIG. 6A) and the second blade 153b may be configured to cut the proximal portion 127 (shown in FIG. 6A) along the cutting region 142b of proximal portion 127 (shown in FIG. 6A).

For example, FIG. 13 illustrates the cutter 150 being translated in a proximal-to-distal direction to initiate cutting of the proximal portion 127 along the cutting regions 142a, 142b (shown in FIG. 6A). Translation of the cutter 150 in a proximal-to-distal direction is depicted by the directional arrow 165 in FIG. 13. FIG. 13 illustrates that translation of the cutter 150 in a proximal-to-distal direction may permit each of the blades 153a, 153b to engage and cut the wall of the proximal portion 127. Additionally, it can be appreciated from FIG. 13 that the access sheath 24 may be configured such the inner surface 163 (shown in FIG. 12) of the hub 168 (whereby the hub 168 may represent any hub disclosed herein, including the hub 26 and hub 126 disclosed herein) may be circumferentially aligned with an inner surface 166 of the proximal portion 127. Accordingly, as the cutter 150 is translated in a proximal-to-distal direction, the top surface of each of the guide members 161a, 161b may contact the inner surface 166 of the proximal portion 127, thereby preventing the first grip member 151a and the second grip member 151b from expanding radially outward and pulling the blades 153a, 153b out of the wall of the proximal portion 127 and thereby allowing the cutting surface 156 of each of the blades 153a, 153b to cut the proximal portion 127.

FIG. 14 shows a cross-sectional view taken along line 14-14 of the expandable portion 28 of the access sheath 24/84 shown in FIGS. 2A-2B. FIG. 14 illustrates the expandable portion 28 in a pre-expanded configuration.

FIG. 14 illustrates the liner 32 including two folded sections 74 extending along the inner surface of the liner 32. While FIG. 14 shows two folded sections 74, it is contemplated that the liner 32 may include more or less than two folded sections 74. For example, the liner 32 may include 1, 2, 4, 5, 6, 7, 8, 9, 10 or more folded sections 74. Further, in some examples, the inner surface of the liner 32 may include one or more layers or coatings, such as a lubricious coating, a hydrophilic coating, a hydrophobic coating, or other suitable coatings, and the like, or liner 32 may include a lubricant disposed thereon.

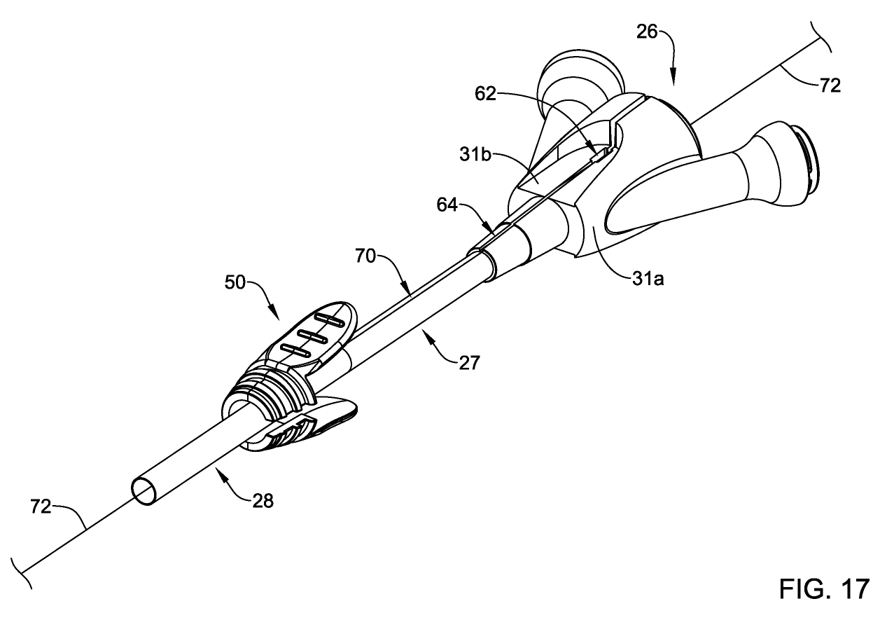

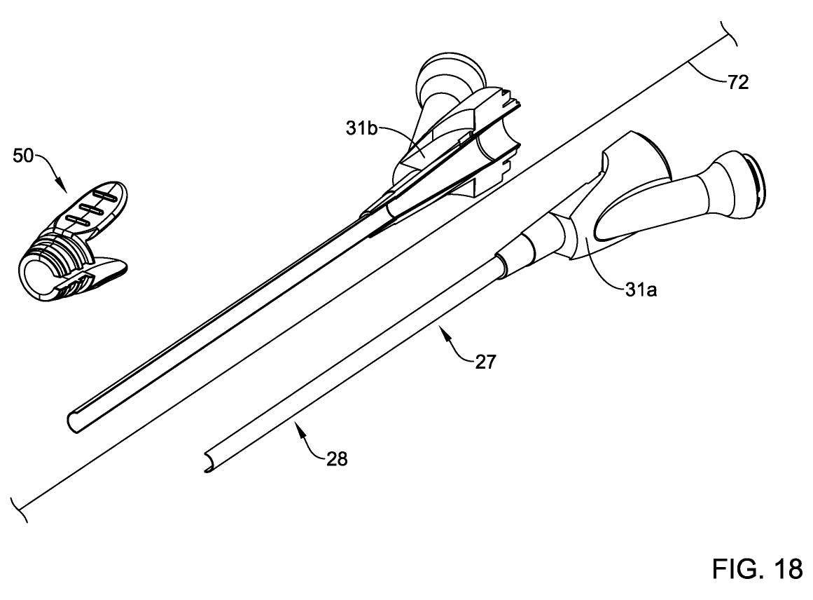

FIG. 14 further illustrates that each folded section 74 may include a folded pleat 76a, 76b which may correspond to a position along the liner 32 in which the liner 32 may be cut, split, peeled, separated, etc. after the liner expands from a pre-expanded configuration to an expanded configuration. In some examples, the cutter 50 may cut the liner 32 along the folded pleat 76a, 76b after the liner expands from a pre-expanded configuration to an expanded configuration (shown in FIG. 18). In other examples, the liner 32 may tear along the folded pleat 76a, 76b as a user peels the proximal portion 127 (as shown in FIG. 19). Actuation of the cutter 50 to cut both the proximal portion 27 and the expandable portion 28 will be described in greater detail with respect to FIGS. 17-18 herein. Peeling the proximal portion 127 along the folded pleat 76a, 76b will be described in greater detail with respect to FIG. 19.

FIG. 14 further illustrates that, in some examples, at least a portion of the outer sheath 31 may be engaged (e.g., attached) to at least a portion of the liner 32. For example, the inner surface of the outer sheath 31 may be engaged to at least a portion of the outer surface of the liner 32. For example, the outer sheath 31 may be bonded, laminated, fused, glued, co-molded, melted, welded, or other suitable means, to a portion of the liner 32. In some examples, outer sheath 31 may be permanently attached to the outer surface of liner 32.

In some examples, the outer sheath 31 may be formed from a polymeric material, which may form at least a portion of a wall of outer sheath 31. In some examples, the outer sheath 31 may be formed from the same material as the liner 32. In some examples, the outer sheath 31 may be formed from a different material than the liner 32. In some examples, some or all of the outer sheath 31 may be fixedly attached to the outer surface of the wall of the liner 32. In some examples, the entire outer sheath 31 may be fixedly attached to the outer surface of the liner 32. However, in some examples, the outer sheath 31 may be free from attachment to all or a portion of the liner 32.

In some examples, at least a portion of the outer sheath 31 may extend continuously around a circumference of the liner 32 in a first configuration. In some examples, at least a portion of the outer sheath 31 may be discontinuous. For example, as discussed herein, the outer sheath 31 may include a plurality of apertures 34 (e.g., openings, cutouts, perforations, notches, holes, or other weakening features) formed in the wall of the outer sheath 31 which effectively remove at least (or in some cases, only) a portion of the wall of outer sheath 31. Further, it is contemplated that the apertures 34 may be any shape. For example, the apertures 34 may be circular, square, rectangular, ovular, triangular, diamond-shaped, polygonal-shaped (e.g., pentagon, hexagon, etc.) or the like. As discussed herein, the plurality of apertures 34 formed in the wall of outer sheath 31 may be arranged in one or more longitudinal lines along the length of the expandable portion 28.

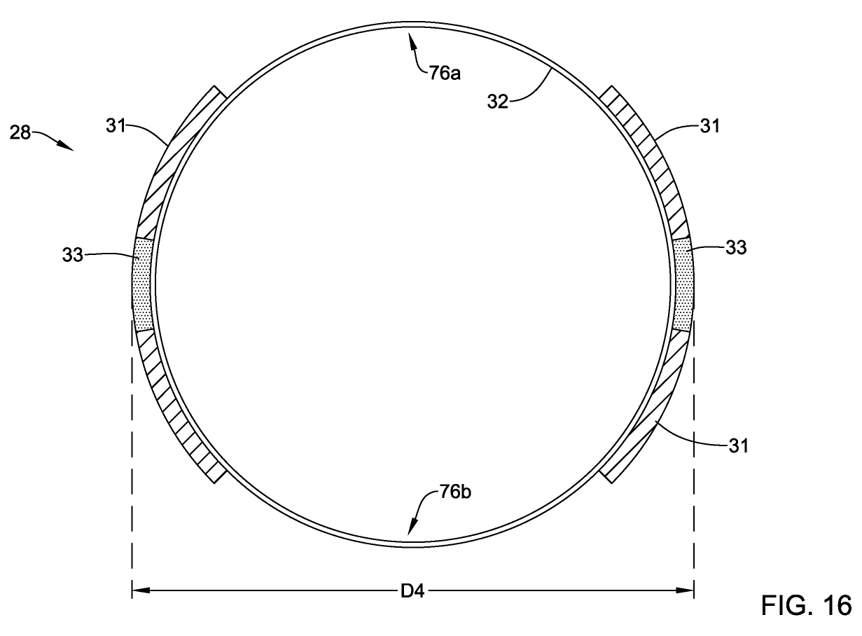

In at least some examples, the apertures 34 (e.g., openings, holes, spaces, etc.) or other apertures formed in the outer sheath 31 may be positioned adjacent to, radially outward of and/or directly over the one or more of the folds 74 formed in the liner 32. For example, FIG. 14 illustrates the apertures 34 positioned adjacent the one or more of the folds 74 in the liner 32. In some instances, the apertures 34 may be adjacent a position along the outer sheath 31 in which one or more of the folds 74 begin to wrap underneath the outer sheath 31. It can be appreciated that the position at which the apertures 34 are positioned relative to a corresponding fold 74 may be the position at which a fold 74 initially begins to expand when the liner 32 shifts from the configuration shown in FIG. 14 to an expanded configuration (shown in FIG. 16).

As discussed herein, the liner 32 may be configured to radially expand from an unexpanded, delivery configuration to an expanded configuration when subjected to a radially outward force from within the liner 32. In at least some examples, the liner 32 may be substantially or completely compliant and/or the liner 32 may have no radial self-bias (e.g., no radially inward self-bias and/or no radially outward self-bias). In other words, the liner 32 may be non-self-supporting and may not include a mechanism to radially expand and/or open on its own (i.e., absent a radially outward force exerted upon liner 32). Instead, the liner 32 may require a device or object that has a greater outer diameter than an inner diameter of the lumen of the liner 32 to be disposed within the lumen of the liner 32 to push and/or force the liner 32 radially outward toward the expanded configuration (shown in FIG. 18).

As discussed above, the liner 32 may include one or more folds 74 formed therein in the non-expanded configuration (shown in FIG. 14). In some examples, the one or more folds 74 may each fold back on themselves to form, for example, a wave shape, an S-shape, T-shape and/or a Z-shape when viewed in cross-section. In some examples, each of the one or more folds forming a T-shape may include two or more distinct waves, S-shaped, or Z-shaped secondary folds within and thus forming each of the one or more T-shaped folds. Other shapes and configurations, while not expressly illustrated, are also contemplated.

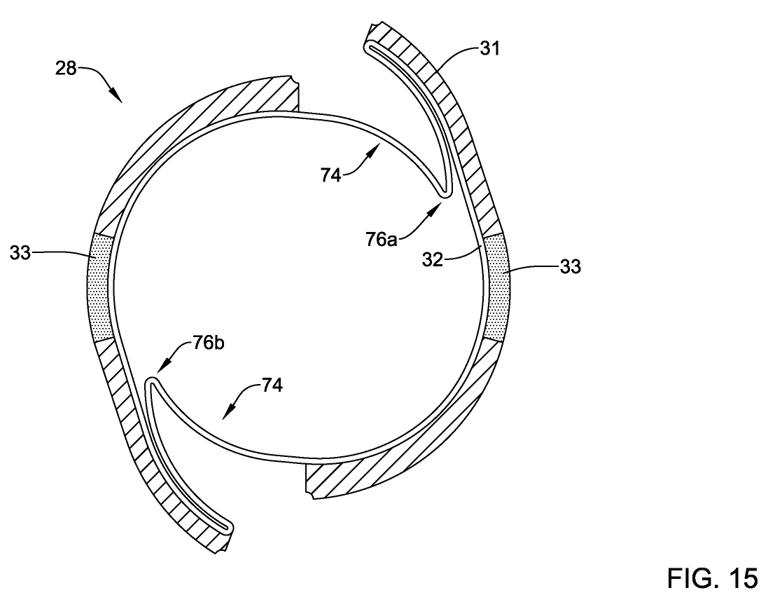

As discussed herein, the liner 32 may be configured to radially expand from a first inner diameter to a second inner diameter. For example, FIGS. 15-16 illustrate the liner 32, and thus the expandable portion 28 of the elongate shaft 25, expanding from a first unexpanded configuration to a second expanded configuration.

FIG. 15 illustrates that the outer sheath 31 may be configured to separate, split, or tear as the liner 32 is expanded from a first configuration to a second, expanded configuration. The expansion may be due to a radially outward force applied from within the liner 32. In some examples, the outer sheath 31 may be configured to separate, split, perforate and/or tear along and/or through the plurality of apertures 34 (or other weakening features formed in the wall of the outer sheath 31). In other words, the outer sheath 31 may separate, split, or tear where the outer sheath 31 is discontinuous and/or constructed of a thinner material (e.g., along apertures 34). As discussed above, as the liner 32 radially expands outward toward an expanded configuration, the outer sheath 31 may tear along preferential tear points corresponding to the apertures 34. Additionally, it can be appreciated the outer sheath 31 and/or the liner 32 may be configured to be atraumatic to the vasculature. For example, during expansion of the expandable portion 28, the geometry and/or general shape of the surfaces of the outer sheath 31 and/or liner 32 may be configured to be atraumatic to the vasculature.

As discussed above, the plurality of apertures 34, or other weakening features formed in the wall of the outer sheath 31 may be positioned adjacent to, radially outward of, in communication with, and/or directly over the one or more folds 74 formed in the liner 32. As mentioned above, in some examples, the plurality of apertures 34, or other weakening features formed in the wall of the outer sheath 31 may be arranged in one or more longitudinal lines along the length of the expandable portion 28. Accordingly, in some examples, the plurality of apertures 34 or other weakening features formed in the wall of the outer sheath 31 may be arranged in one or more longitudinal lines adjacent to, radially outward of, in communication with, and/or directly over the one or more folds 74 formed in the liner 32.

FIG. 16 illustrates the expandable portion 28 after having been expanded to an expanded configuration. FIG. 16 illustrates that the expandable portion 28 may have a diameter D₄in the expanded configuration. In some examples, diameter D₄ may be about 0.200 inches (5.08 millimeters) to about 0.250 inches (6.35 millimeters). It can be appreciated that the diameter D₄ may represent the diameter of the expandable portion 28 taken at any position along the expandable portion 28 when in the expanded configuration. Accordingly, it can be appreciated that the diameter D₄ of the expanded, expandable portion 28 may be substantially equivalent to the diameter D₂ (shown in FIGS. 2A-2B) and/or the diameter D₃(shown in FIG. 4) of the proximal portion 27. Additionally, it can be appreciated the outer sheath 31 and/or the liner 32 may be configured to be atraumatic to the vasculature. For example, after expansion of the expandable portion 28 (such as the configuration illustrated in FIG. 16), the geometry and/or general shape of the surfaces of the outer sheath 31 and/or liner 32 may be configured to be atraumatic to the vasculature.

FIG. 16 further illustrates the relative position of the folded pleats 76a, 76b after the liner 32 has expanded to the expanded configuration. It can be appreciated that, when in the expanded configuration shown in FIG. 16, each of the folded pleats 76a, 76b may circumferentially align with the frangible regions 142a, 142b (shown in FIG. 6), respectively, of the proximal portion 27.

Additionally, in some examples, after expansion (such as the configuration illustrated in FIG. 16), the expandable portion 28 may recover (e.g., relax, contract, refold, fold, etc.) to a configuration similar to that shown in FIG. 15, whereby the outer diameter of the expandable portion 28 is less than that shown in the expanded configuration shown in FIG. 16. The ability for the expandable portion 28 to reduce its diameter to a configuration which is less than that shown in the expanded configuration shown in FIG. 16 may be beneficial to improve blood flow and/or reduce restriction and leg ischemia risk by having a smaller cross-sectional area. In some examples, the reduction in outer diameter and cross-sectional area may occur as the result of the liner 32 folding back on itself in one or more locations.

FIG. 17 is a perspective view of the cutter 50 being translated in a proximal-to-distal direction to cut both the proximal portion 27 along the frangible region 142a (shown in FIG. 6A) and the expandable portion along the folded pleat 76a (shown in FIGS. 14-16). For clarity purposes, the supporting members 33 have been omitted from FIGS. 17-18. As discussed herein, it can be appreciated that, when in the expanded configuration (shown in FIG. 16), each of the folded pleats 76a, 76b may circumferentially align with the frangible regions 142a, 142b (shown in FIG. 6), respectively, of the proximal portion 27. Accordingly, it can be further appreciated that actuation of the cutter 50 in a distal direction may initially cut the frangible regions 142a,142b of the proximal portion (it is noted that the frangible region 142b is hidden from view in FIG. 17) and then proceed to cut the expanded liner 32 along the folded pleats 76a, 76b (it is noted that the frangible region 142b is hidden from view in FIG. 17), thereby cutting the entirety of the elongate shaft 25 apart and permitting the access sheath to be removed from the guidewire 72 (or other portion of a medical device such as the blood pump 10 shown in FIG. 1). Reference number 70 depicts the cutting surface 56 of the blade 53 to engage and cut the wall of the proximal portion 27 along the frangible region 142a, while reference numeral 78 represents the folded pleat 76a along the liner 32 which is longitudinally aligned with the frangible region 142a and is in the process of being cut by the cutter 50 in FIG. 17.

As discussed herein, FIG. 18 illustrates the separation of the proximal portion 27 and the separation of the expandable portion 28 (after being cut as discussed herein). Further, FIG. 18 illustrates the first hub member 31a separated from the second hub member 31b. As discussed herein, the first hub member 31a and the second hub member 31b may be releasably attached to one another via a splitable membrane 60 (shown in FIG. 9). In some examples, the splitable membrane 60 attaching the first hub member 31a and the second hub member 31b may be formed from a soft durometer material which may be configured to split when the first hub member 31a is pulled away from the second hub member 31b. It can be further appreciated that separating the first hub member 31a from the second hub member 31b may permit the hub 26 to be removed from the guidewire 72 or other portion of a medical device such as the proximal catheter portion 16 of the blood pump 10 shown in FIG. 1.

As discussed herein, FIG. 19 illustrates the access sheath 84 including the splitable hub 77, the proximal portion 27 and the expandable portion 28. It can be appreciated that the proximal portion 27 shown in FIG. 19 may be similar in form and function to the proximal portion 127 shown in FIGS. 6A-6B. For example, the proximal portion 27 shown in FIG. 19 may include the first frangible region 142a and the second frangible region 142b shown in FIGS. 6A-6B.

Additionally, FIG. 19 illustrates splitting the first hub member 78a and the second hub member 78b of the hub 77 (shown in FIG. 2B) and splitting (e.g., peeling, separating) the proximal portion 27 along both the first frangible region 142a and the second frangible region 142b. It can be appreciated that splitting the proximal portion 27 along both the first frangible region 142a and the second frangible region 142b may split the proximal portion 27 into a first proximal portion 88 and a second proximal portion 90. Further, in the expanded configuration (shown in FIG. 16), each of the folded pleats 76a, 76b may circumferentially align with the frangible regions 142a, 142b (shown in FIG. 6A-6B), respectively, of the proximal portion 27. Accordingly, it can be further appreciated that splitting the proximal portion 27 may initially occur along the frangible regions 142a, 142b of the proximal portion 27, whereby the splitting of the proximal portion 27 may proceed to tearing the expanded liner 32 along the folded pleats 76a, 76b, thereby separating the entirety of the elongate shaft 25 (shown in FIG. 2B) apart and permitting the access sheath 84 (shown in FIG. 2B) to be removed from the guidewire 72 or other portion of a medical device such as the proximal catheter portion 16 of the blood pump 10 shown in FIG. 1.

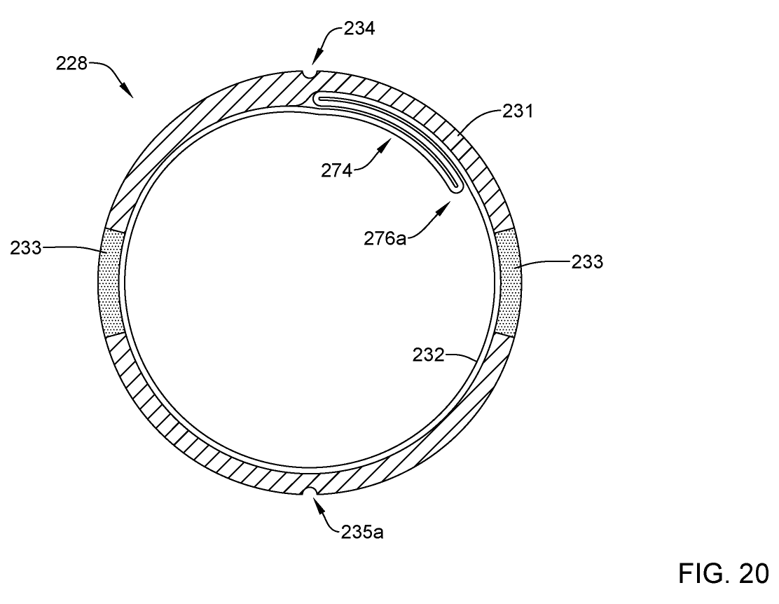

FIG. 20 illustrates a cross-sectional view of an example expandable portion 228 of an access sheath in a pre-expanded configuration. The expandable portion 228 may be similar in form and function to the expandable portion 28 described herein. For example, the expandable portion 228 may include an outer sheath 231 generally surrounding an inner liner 232 and defining a central lumen extending therethrough. Additionally, FIG. 20 illustrates that the expandable portion 228 may further include one or more support members 233 (e.g., spines) positioned between the outer sheath 231 and the liner 232 and extending longitudinally along at least a portion of the expandable portion 228. The support members 233 may be configured to provide structural support to the expandable portion 228.

FIG. 20 further illustrates that the outer sheath 231 may include a preferential separation region, such as one or more apertures 234 (e.g., openings, cutouts, perforations, notches, holes, or other weakening features) extending through at least a portion of the wall of the outer sheath 231. In some examples, the apertures 234 may be arranged or aligned longitudinally along the length of the expandable portion 228. In some examples, the apertures 234 may be positioned between adjacent support members 233.

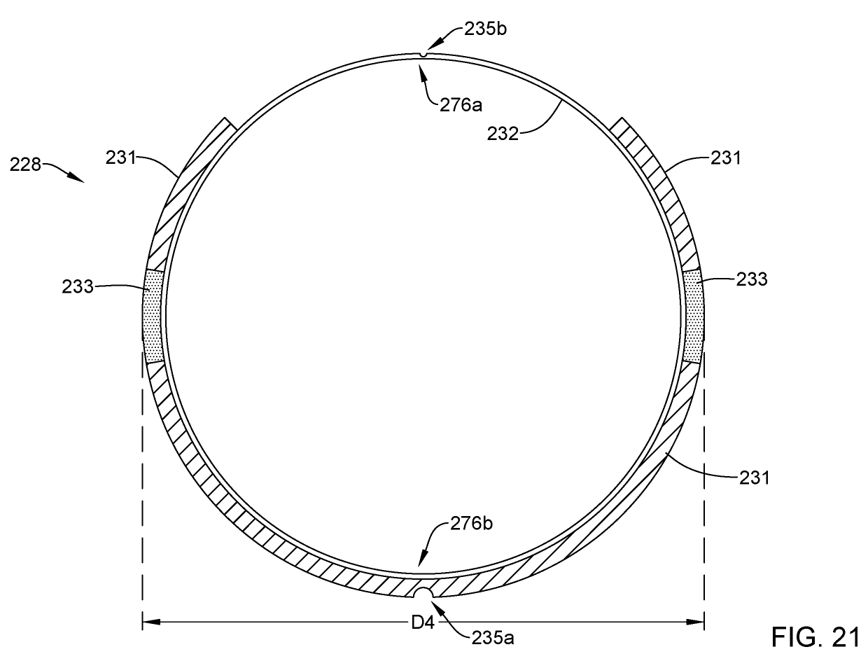

FIG. 20 further illustrates the liner 232 including a fold 274. While FIG. 20 illustrates a single fold 274, it is contemplated that the liner 232 may include more or fewer folds, in other examples. The fold 274 may include a folded pleat 276a which may correspond to a position along the liner 232 in which the liner 232 may be cut, split, peeled, separated, or otherwise opened after the liner 232 expands from a pre-expanded configuration (shown in FIG. 20) to an expanded configuration (shown in FIG. 21).

In some examples, the outer sheath 231 may further include a frangible region 235a formed along the outer sheath 231. The frangible region 235a may define a region of the outer sheath 231 configured to fracture, tear, or otherwise separate when subjected to a threshold pull-away or peel force. In some examples, the frangible region 235a may be positioned generally opposite or approximately 180 degrees from the fold 274 of the liner 232. Further, in some examples, the frangible region 235a may extend radially inward from an outer surface of the outer sheath 231. In some examples, the frangible region 235a may include a series of indentations (e.g., perforations) which may be aligned with one another along the central longitudinal axis of the expandable portion 228. Similarly, the frangible region 235a may include a series of indentations which may be aligned with one another along a central longitudinal axis of the expandable portion 228.

However, in other examples, the frangible region 235a may not include indentations or perforations. Instead, the frangible region 235a may be defined by a region of one or more of the outer sheath 231 and the liner 232 having a reduced wall thickness and/or a reduced outer diameter relative to adjacent portions of the expandable portion 228 (e.g., score line, weakened area, groove, etc.), thereby defining a region configured to preferentially separate when a threshold pull-away force is applied

FIG. 21 illustrates a cross-sectional view of the expandable portion 228 after the liner 232 has expanded to an expanded configuration. As shown in FIG. 21, the expandable portion 228 may have an expanded diameter D4 similar to the expanded diameter D4 of the expandable portion 28 illustrated in FIG. 16. In some examples, the diameter D4 may be about 0.200 inches (5.08 millimeters) to about 0.250 inches (6.35 millimeters). FIG. 21 further illustrates the position of the folded pleat 276a of the fold 274 after the liner 232 has expanded to the expanded configuration. In some examples, the folded pleat 276a may correspond to a region along which the liner 232 may be torn or separated during removal of the access sheath. For example, FIG. 21 illustrates that the outer sheath 231 may include the frangible region 235a along the outer sheath 231 and an additional frangible region 235b positioned along a portion of the liner 232. In some examples, the frangible region 235b may be positioned adjacent to or otherwise coincide with the folded pleat 276a described herein.

Additionally, the frangible regions 235a, 235b may correspond to regions of the outer sheath 231 that are configured to separate when subjected to a threshold pull-away or peel force. For example, the frangible regions 235a, 235b of the expandable portion 228 may be longitudinally aligned with the frangible regions 142a, 142b (shown in FIGS. 6A-6B) of the proximal portion 127 (shown in FIGS. 6A-6B), respectively. Accordingly, it can be appreciated that when the hub (e.g., hub 77 shown in FIG. 2B) is split, the proximal portion 127 may initially be peeled along the frangible regions 142a, 142b. Continued peeling of the access sheath 84 (shown in FIG. 2B) may proceed distally along the frangible regions 235a, 235b of the expandable portion 228, thereby separating the expandable portion 228 into two pieces and permitting the access sheath 84 to be removed from a guidewire or other portion of a medical device extending through the access sheath 84. This process of peeling splitting the hub 77 and peeling along the frangible regions 142a, 142b, 235a, 235b may be similar to that illustrated and described with respect to FIG. 19.

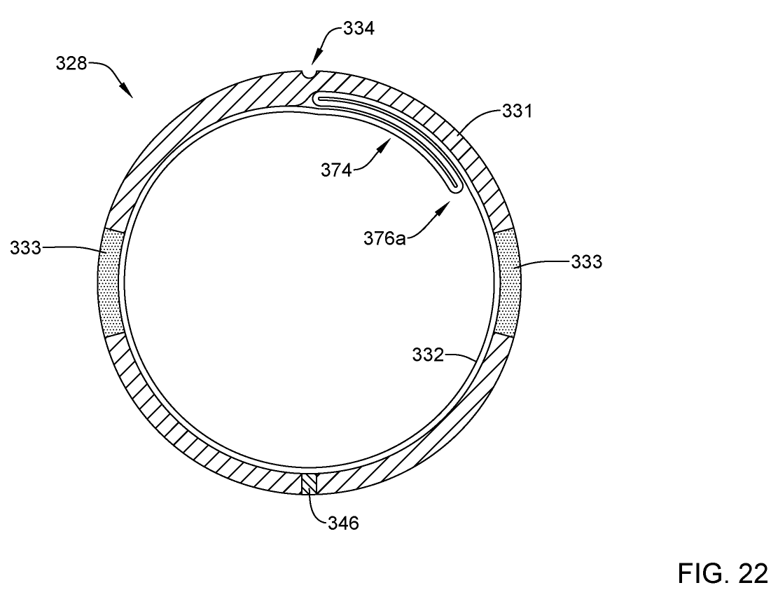

FIG. 22 illustrates a cross-sectional view of an example expandable portion 328 of an access sheath in a pre-expanded configuration. The expandable portion 328 may be similar in form and function to the expandable portion 28 described herein. For example, the expandable portion 328 may include an outer sheath 331 generally surrounding an inner liner 332 and defining a central lumen extending therethrough. Additionally, FIG. 22 illustrates that the expandable portion 328 may further include one or more support members 333 (e.g., spines) positioned between the outer sheath 331 and the liner 332 and extending longitudinally along at least a portion of the expandable portion 328. The support members 333 may be configured to provide structural support to the expandable portion 328.

FIG. 22 further illustrates that the outer sheath 331 may include a preferential separation region, such as one or more apertures 334 (e.g., openings, cutouts, perforations, notches, holes, or other weakening features) extending through at least a portion of the wall of the outer sheath 331. In some examples, the apertures 334 may be arranged or aligned longitudinally along the length of the expandable portion 328. In some examples, the apertures 334 may be positioned between adjacent support members 333.

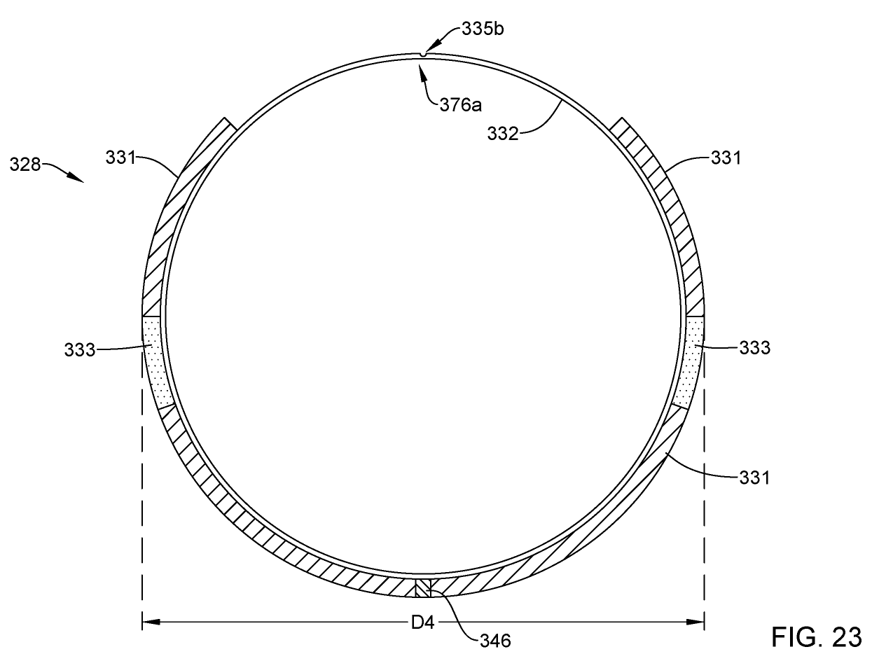

FIG. 22 further illustrates the liner 332 including a fold 374. While FIG. 22 illustrates a single fold 374, it is contemplated that the liner 332 may include more or fewer folds, in other examples. The fold 374 may include a folded pleat 376a which may correspond to a position along the liner 332 in which the liner 332 may be cut, split, peeled, separated, or otherwise opened after the liner 332 expands from a pre-expanded configuration (shown in FIG. 22) to an expanded configuration (shown in FIG. 23).

In some examples, the outer sheath 331 may further include a frangible striped extrusion material 346 formed along the outer sheath 331. The frangible striped extrusion material 346 may define a preferential separation region of the outer sheath 331 configured to fracture, tear, or otherwise separate when subjected to a threshold pull-away or peel force. In some examples, the frangible striped extrusion material 346 may be formed from a material different from the material used to form the outer sheath 331. For example, the frangible striped extrusion material 346 may include a material having a lower strength, reduced wall thickness, or otherwise different mechanical properties than the material forming the outer sheath 331 such that the striped extrusion material 346 defines a preferential separation region. In some examples, the frangible striped extrusion material 346 may be positioned generally opposite or approximately 180 degrees from the fold 374 of the liner 332.

In some examples, the outer sheath 331 and the frangible striped extrusion material 346 may be formed together during a co-extrusion manufacturing process. For example, the outer sheath 331 may be extruded from a first material while the frangible striped extrusion material 346 may be simultaneously extruded from a second material such that the striped extrusion material 346 extends longitudinally along the outer sheath 331 and defines a weakened region configured to preferentially separate when subjected to a threshold pull-away or peel force.

FIG. 23 illustrates a cross-sectional view of the expandable portion 328 after the liner 332 has expanded to an expanded configuration. As shown in FIG. 23, the expandable portion 328 may have an expanded diameter D4 similar to the expanded diameter D4 of the expandable portion 28 illustrated in FIG. 16. In some examples, the diameter D4 may be about 0.200 inches (5.08 millimeters) to about 0.250 inches (6.35 millimeters). FIG. 23 further illustrates the position of the folded pleat 376a of the fold 374 after the liner 332 has expanded to the expanded configuration. In some examples, the folded pleat 376a may correspond to a preferential separation region along which the liner 332 may be torn or separated during removal of the access sheath.

Additionally, FIG. 23 illustrates that the outer sheath 331 may include the frangible striped extrusion material 346 along the outer sheath 331 and an additional frangible region 335b positioned along a portion of the liner 332. In some examples, the frangible region 335b may be positioned adjacent to or otherwise coincide with the folded pleat 376a described herein.

Additionally, the frangible striped extrusion material 346 and the frangible region 335b may correspond to regions of the outer sheath 331 configured to separate when subjected to a threshold pull-away or peel force. For example, the frangible striped extrusion material 346 and the frangible region 335b of the expandable portion 328 may be longitudinally aligned with the frangible regions 142a, 142b (shown in FIGS. 6A–6B) of the proximal portion 127 (shown in FIGS. 6A–6B), respectively. Accordingly, it can be appreciated that when the hub (e.g., hub 77 shown in FIG. 2B) is split, the proximal portion 127 may initially be peeled along the frangible regions 142a, 142b. Continued peeling of the access sheath 84 (shown in FIG. 2B) may proceed distally along the frangible striped extrusion material 346 and the frangible region 335b of the expandable portion 328, thereby separating the expandable portion 328 into two pieces and permitting the access sheath 84 to be removed from a guidewire or other portion of a medical device extending through the access sheath. This process of peeling splitting the hub 77 and peeling along the frangible regions 142a, 142b, the frangible striped extrusion material 346 and the frangible region 335b may be similar to that illustrated and described with respect to FIG. 19.

In some examples, access sheath 24 (or other access sheaths and components thereof described herein) may be made from materials such as metals, metal alloys, polymers, ceramics, metal-polymer composites, or other suitable materials, and the like. Some examples of suitable materials may include metallic materials such as stainless steels (e.g. 304v stainless steel or 316L stainless steel), nickel-titanium alloys (e.g., nitinol, such as super elastic or linear elastic nitinol), nickel-chromium alloys, nickel-chromium-iron alloys, cobalt alloys, nickel, titanium, platinum, or alternatively, a polymeric material, such as a high performance polymer, or other suitable materials, and the like. The word nitinol was coined by a group of researchers at the United States Naval Ordinance Laboratory (NOL) who were the first to observe the shape memory behavior of this material. The word nitinol is an acronym including the chemical symbol for nickel (Ni), the chemical symbol for titanium (Ti), and an acronym identifying the Naval Ordinance Laboratory (NOL).

In some examples, the access sheath 24 (or other access sheaths and components thereof described herein) may be made from materials such as, for example, a polymeric material, a ceramic, a metal, a metal alloy, a metal-polymer composite, or the like. Examples of suitable polymers may include nylons, acrylonitrile butadiene styrene (ABS), polyurethane, a polyether-ester such as ARNITEL® available from DSM Engineering Plastics, a polyester such as HYTREL® available from DuPont, a linear low density polyethylene such as REXELL®, a polyamide such as DURETHAN® available from Bayer or CRISTAMID® available from Elf Atochem, an elastomeric polyamide, a block polyamide/ether, a polyether block amide such as PEBA available under the trade name PEBAX®, silicones, polyethylene, Marlex high-density polyethylene, polyetheretherketone (PEEK), polyimide (PI), and polyetherimide (PEI), a liquid crystal polymer (LCP) alone or blended with other materials. In some examples, a suitable polymeric material may have a yield strain of at least 20%, at least 30%, at least 40%, at least 50%, or more. In some examples, the sheath, the membrane, the liner or other components of the access sheaths disclosed herein may be made from a material having a low coefficient of friction. In some examples, the sheath, the membrane, the liner or other components of the access sheaths disclosed herein may be formed from a fluoropolymer, such as polytetrafluoroethylene (PTFE) or fluorinated ethylene propylene (FEP).

Portions of access sheath 24 (or other access sheaths and components thereof described herein) may be made of, may be doped with, may include a layer of, or otherwise may include a radiopaque material. Radiopaque materials are understood to be materials capable of producing a relatively bright image on a fluoroscopy screen or another imaging technique such as X-ray during a medical procedure. This relatively bright image aids the user of device in determining its location. For example, one or more of the elements described above (i.e., the sheath, the membrane, the medical device, etc.) may include or be formed from a radiopaque material. Suitable materials can include, but are not limited to, bismuth subcarbonate, iodine, gold, platinum, palladium, tantalum, tungsten or tungsten alloy, and the like.

It should be understood that although the above discussion was focused on percutaneous medical procedures within the vasculature of a patient, other examples or methods in accordance with the disclosure can be adapted and configured for use in other parts of the anatomy of a patient. For example, devices and methods in accordance with the disclosure can be adapted for use in the digestive or gastrointestinal tract, such as in the mouth, throat, small and large intestine, colon, rectum, and the like. For another example, devices and methods can be adapted and configured for use within the respiratory tract, such as in the mouth, nose, throat, bronchial passages, nasal passages, lungs, and the like. Similarly, the devices and methods described herein with respect to percutaneous deployment may be used in other types of surgical procedures as appropriate. For example, in some examples, the devices may be deployed in a non-percutaneous procedure. Devices and methods in accordance with the disclosure can also be adapted and configured for other uses within the anatomy.

It should be understood that this disclosure is, in many respects, only illustrative. Changes may be made in details, particularly in matters of shape, size, and arrangement of steps without exceeding the scope of the disclosure. The disclosure’s scope is, of course, defined in the language in which the appended claims are expressed.

What is claimed is:

1. An access sheath for a medical device, comprising:
a hub attached to an elongate shaft, wherein the elongate shaft includes a proximal portion and an expandable portion, wherein the expandable portion is configured to shift between an unexpanded configuration and an expanded configuration;
wherein the proximal portion includes a first diameter;
wherein the expandable portion includes a second diameter in the unexpanded configuration, and wherein the second diameter is less than the first diameter.

2. The access sheath of claim 1, wherein expandable region includes a third diameter in the expanded configuration, and wherein the third diameter is equal to the first diameter.

3. The access sheath of claim 1, wherein the expandable portion includes a distal end, a proximal end and a length extending between the distal end and the proximal end, and wherein at least a portion of the expandable portion tapers along the length.

4. The access sheath of claim 3, wherein the expandable portion includes an inner liner extending along a length of the expandable portion, the inner liner including at least one folded portion extending along the length of the expandable portion.

5. The access sheath of claim 4, wherein the at least one folded portion allows the inner liner to radially expand.

6. The access sheath of claim 5, wherein the at least one folded portion wraps along an inner surface of the inner liner.

7. The access sheath of claim 6, wherein the at least one folded portion includes a first folded pleat and a second folded pleat, wherein the first folded pleat and the second folded pleat extends along the length of the expandable portion.

8. The access sheath of claim 7, wherein the proximal portion includes a first frangible region and a second frangible region extending along a length of the proximal portion, and wherein the first frangible region is longitudinally aligned with the folded pleat, wherein the second frangible region is longitudinally aligned with the second folded pleat when the expandable portion is in the expanded configuration.

9. The access sheath of claim 8, wherein the proximal portion is configured to split along the first frangible region and the second frangible region.

10. The access sheath of claim 9, wherein the hub includes a first hub member configured to separate from a second hub member.

11. The access sheath of claim 10, wherein the separation of the first hub member from the second hub member initiates splitting the proximal portion along the first frangible region and the second frangible region.

12. The access sheath of claim 11, wherein splitting of the proximal portion permits the proximal portion to be removed from a medical device extending within a lumen of the proximal portion.

13. The access sheath of claim 10, wherein the first hub member includes a grip member extending away from the first hub member.

14. The access sheath of claim 1, wherein the expandable portion is configured to shift from the expanded configuration to a recovered configuration after the expandable portion has been expanded to the expanded configuration, and wherein the diameter of the expanded portion in the recovered configuration is less than the third diameter.

15. An access sheath for a medical device, comprising:
a hub attached to an elongate shaft, wherein the elongate shaft includes a proximal portion and an expandable portion;
wherein the proximal portion includes a plurality of support members spaced from one another along a length of the proximal portion;
wherein the expandable portion includes an inner liner having a plurality of folds disposed along an inner surface of the liner;
wherein the expandable portion is configured to shift between a first unexpanded configuration and an expanded configuration;
wherein the proximal portion includes a first diameter in a first configuration;
wherein the expandable portion includes a second diameter in the unexpanded configuration, and wherein the second diameter is less than the first diameter.

16. The access sheath of claim 15, wherein expandable region includes a third diameter in the expanded configuration, and wherein the third diameter is substantially equal to the first diameter.

17. The access sheath of claim 16, wherein the expandable portion includes a distal end, a proximal end and a length extending between the distal end and the proximal end, and wherein the expandable portion tapers along the length from the distal end to the proximal end.

18. The access sheath of claim 17, wherein the proximal portion includes a first frangible region and a second frangible region extending along a length of the proximal portion, and wherein the proximal portion is configured to split along the first frangible region and the second frangible region.

19. The access sheath of claim 18, wherein the hub includes a first hub member configured to separate from a second hub member.

20. An access sheath for a medical device, comprising:
a splitable hub attached to an elongate shaft, wherein the elongate shaft includes a proximal portion and an expandable portion;
wherein the expandable portion includes an inner liner having a plurality of folds extending along an inner surface of the liner, the inner liner configured to shift from an unexpanded configuration to an expanded configuration;
wherein the proximal portion includes a first diameter;
wherein the expandable portion includes a second diameter in the unexpanded configuration and a third diameter in the expanded configuration;
wherein the second diameter is less than the first diameter;
wherein the third diameter is substantially equal to the first diameter.
